(19) United States
(12) Patent Application Publication
(10) Pub. No.: US 2018/0353495 A1
(43) Pub. Date: Dec. 13, 2018

(54) PHARMACEUTICAL COMBINATIONS COMPRISING A PI3K INHIBITOR FOR THE TREATMENT OF CANCER

(71) Applicant: Novartis AG, Basel (CH)

(72) Inventors: Cristian MASSACESI, Neuilly Sur-Seine (FR); Marie-Caroline GERMA, Short Hills, NJ (US)

(21) Appl. No.: 16/106,345

(22) Filed: Aug. 21, 2018

Related U.S. Application Data
(63) Continuation of application No. 15/116,941, filed on Aug. 5, 2016, now abandoned.

(30) Foreign Application Priority Data
Feb. 11, 2014 (EP) 14305179.5

(51) Int. Cl. A61K 31/4439; A61K 31/436; A61K 31/5685; A61K 45/06

(52) U.S. Cl. CPC A61K 31/4439; A61K 2300/00; A61K 2121/00; A61K 45/06; A61K 31/5685; A61K 31/436

(57) ABSTRACT

A pharmaceutical combination comprising: (a) an alpha-isoform specific phosphatidylinositol-3-kinase inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor and (c) exemestane or any pharmaceutically acceptable salt thereof, particularly for use in the treatment or prevention of a proliferative disease; uses of such a combination in the preparation of a medicament for the treatment or prevention of a proliferative disease; pharmaceutical compositions of the combination of said therapeutic agents and methods of treating a proliferative disease in a subject comprising administering to said subject a therapeutically effective amount of such a combination.

### FIELD OF THE INVENTION

A pharmaceutical combination comprising: (a) an alpha-isoform specific phosphatidylinositol-3-kinase inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor and (c) exemestane or any pharmaceutically acceptable salt thereof, particularly for use in the treatment or prevention of a proliferative disease; uses of such a combination in the preparation of a medicament for the treatment or prevention of a proliferative disease; pharmaceutical compositions of the combination of said therapeutic agents and methods of treating a proliferative disease in a subject comprising administering to said subject a therapeutically effective amount of such a combination.

### BACKGROUND OF THE INVENTION

Phosphatidylinositol 3-kinases (PI-3 kinase or PI3K) comprise a family of lipid and serine/threonine kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP3) that, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p85β or p55β. The Class 1B sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)).

PI3K inhibitors are useful therapeutic compounds for the treatment of various conditions in humans. Aberrant regulation of PI3K is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110α isoform, PIK3CA, and for Akt are amplified and increased protein expression of their gene products has been demonstrated in several human cancers. Furthermore, mutations and translocation of p85α that serve to up-regulate the p85-p110 complex have been described in a few human cancers. Finally, somatic missense mutations in PIK3CA that activate downstream signaling pathways have been described at significant frequencies in a wide diversity of human cancers (Kang et al., Proc. Natl. Acad. Sci. USA 102:802 (2005); Samuels et al., Science 304:554 (2004); Samuels et al., Cancer Cell 7:561-573(2005)). These observations show that deregulation of phosphoinositol-3 kinase and the upstream and downstream components of this signaling pathway is one of the most common deregulations associated with human cancers and proliferative diseases (Parsons et al., Nature 436:792(2005); Hennessey at el., Nature Rev. Drug Dis. 4:988-1004 (2005)).

Treatment with mTOR inhibitors has proven efficacious in both advanced renal cell carcinoma and pancreatic neuroendocrine tumors, and recent clinical evidence demonstrates that the mTOR inhibitor everolimus in combination with aromatase inhibitor exemestane results in statistically significant and clinically meaningful improvement in clinical outcome in hormone receptor positive, HER2-negative breast cancer. However, clinical results show that patients develop resistance to mTORC1 inhibition and lack of response and/or progression to mTOR inhibitors occurs. In spite of numerous treatment options for cancer patients, there remains a significant unmet need for effective and safe therapeutic agents for cancer treatment and a need for their preferential use in combination therapy. (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) is a novel compound that highly selectively inhibits the activity of the alpha(α)-isoform of phosphatidylinositol 3-kinase. These specific alpha-isoform specific PI3K inhibitors are believed to have a strong beneficial interaction (e.g, synergistic) and/or improved anti-proliferative activity when used in combination with an mTOR inhibitor (particularly everolimus) and the aromatase inhibitor exemestane. It is therefore an object of the present invention to provide for a medicament to improve treatment of cancer.

### SUMMARY OF THE INVENTION

The present invention relates to a pharmaceutical combination comprising: (a) an alpha-isoform specific phosphatidylinositol-3-kinase (PI3K) inhibitor (S)-Pyrrolidine-1,2-dicarboxylic acid 2-amide 1-({4-methyl-5[-2-(2,2,2-trifluoro-1,1-dimethyl-ethyl)-pyridin-4-yl]-thiazol-2-yl}-amide) or any pharmaceutically acceptable salt thereof, (b) an mTOR inhibitor and (c) exemestane or any pharmaceutically acceptable salt thereof, particularly for separate, simultaneous or sequential use for the treatment or prevention of a proliferative disease.

In a preferred embodiment, the present invention relates to a pharmaceutical combination comprising (a) an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor everolimus or any pharmaceutically acceptable salt thereof, and (c) exemestane or any pharmaceutically acceptable salt thereof, particularly for use in the treatment or prevention of a hormone-receptor positive breast cancer.

In a further embodiment, the present invention relates to a method of treating or preventing a proliferative disease in a subject comprising administering to said subject a therapeutically effective amount of a COMBINATION OF THE INVENTION.

In a further embodiment, the present invention relates to the use of a COMBINATION OF THE INVENTION for the preparation of a pharmaceutical composition or medicament for the treatment or prevention of a proliferative disease.

In a further embodiment, the present invention relates to the use of a COMBINATION OF THE INVENTION for the treatment or prevention of a proliferative disease.

In a further embodiment, the present invention relates to a pharmaceutical composition or combined preparation, comprising a quantity of COMBINATION OF THE INVENTION which is jointly therapeutically effective against a proliferative disease, and optionally at least one pharmaceutically acceptable carrier.

In a further embodiment, the present invention relates to a combined preparation comprising (a) one or more dosage units of an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof and (b) one or more dosage units of an mTOR inhibitor and (c) one or more dosage units of exemestane or any pharmaceutically acceptable salt thereof, for use in the treatment or prevention of a proliferative disease.

In a further embodiment, the present invention provides a commercial package comprising as active ingredients of COMBINATION OF THE INVENTION, together with instructions for simultaneous, separate or sequential administration of said combination to a patient in need thereof for use in the treatment or prevention of a proliferative disease.

In a further embodiment, the present invention provides a commercial package comprising as active ingredient an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and instructions for simultaneous, separate or sequential administration of said active ingredient with an mTOR inhibitor everolimus or any pharmaceutically acceptable salt thereof and exemestane or any pharmaceutically acceptable salt thereof to a patient in need thereof for use in the treatment or prevention of a proliferative disease.

### DETAILED DESCRIPTION OF THE INVENTION

The present invention relates to a pharmaceutical combination comprising: (a) an alpha-isoform specific phosphatidylinositol-3-kinase (PI3K) inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor and (c) exemestane or any pharmaceutically acceptable salt thereof, particularly for separate, simultaneous or sequential use for the treatment or prevention of a proliferative disease.

The general terms used herein are defined with the following meanings, unless explicitly stated otherwise:

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “combination” or “pharmaceutical combination” as used herein defines either a fixed combination in one dosage unit form or a kit of parts for the combined administration where the therapeutic agents may be administered independently at the same time or separately within time intervals that allow that the therapeutic agents show a cooperative, e.g., synergistic, effect.

The term “combined administration” as used herein is defined to encompass the administration of the selected therapeutic agents to a single patient, and are intended to include treatment regimens in which the therapeutic agents are not necessarily administered by the same route of administration or at the same time.

The term “fixed combination” means that the therapeutic agents are administered to a patient simultaneously in the form of a single entity or dosage form.

The term “a combined preparation” is defined herein to refer to especially a “kit of parts” in the sense that the therapeutic agents (a), (b) and (c) as defined above can be dosed independently or by use of different fixed combinations with distinguished amounts of the therapeutic agents (a), (b) and (c) simultaneously or at different time points. The parts of the kit of parts can then, e.g., be administered simultaneously or chronologically staggered, that is at different time points and with equal or different time intervals for any part of the kit of parts. The ratio of the total amounts of the therapeutic agent (a) to the therapeutic agent (b) to the therapeutic agent (c) to be administered in the combined preparation can be varied, e.g., in order to cope with the needs of a patient sub-population to be treated or the needs of the single patient.

The term “pharmaceutically acceptable” is defined herein to refer to those compounds, materials, biologic agents, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

The term “pharmaceutical composition” is defined herein to refer to a mixture or solution containing at least one therapeutic agent to be administered to a subject, e.g., a mammal or human, in order to prevent or treat a particular disease or condition affecting the mammal.

The term “phosphatidylinositol 3-kinase inhibitor” or “PI3K inhibitor” is defined herein to refer to a compound or biologic agent which selectively targets, decreases or inhibits the phosphatidylinositol 3-kinase.

The term “mammalian target of rapamycin inhibitor” or “mTOR inhibitor” as used herein refers to a compound or biologic agent which targets, decreases or inhibits the activity/function of serine/theronine mTOR kinase.

The term “aromatase inhibitor” as used herein refers to a compound or biologic agent which inhibits the estrogen production, i.e. the conversion of the substrates androstenedione and testosterone to estrone and estradiol, respectively.

The term “treating” or “treatment” as used herein comprises a treatment relieving, reducing or alleviating at least one symptom in a subject or effecting a delay of progression of a proliferative disease, particularly a cancer. For example, treatment can be the diminishment of one or several symptoms of a proliferative disease or complete eradication of a proliferative disease. Within the meaning of the present invention, the term “treat” also denotes to arrest, delay the onset (i.e., the period prior to clinical manifestation of a proliferative disease) and/or reduce the risk of developing or worsening a proliferative disease. The term “prevention” is used herein to mean prevent, delay or treat, or all, as appropriate, development or continuance or aggravation of a proliferative disease in a subject.

The term “joint therapeutic effect” or “jointly therapeutically effective” means that the therapeutic agents of the combination may be given separately (in a chronologically staggered manner, especially a sequence-specific manner) in such time intervals that they prefer, in the warm-blooded animal, especially human, to be treated, still show a (preferably synergistic) interaction (joint therapeutic effect). Whether this is the case can, inter alia, be determined by following the blood levels, showing that both or all therapeutic agents are present in the blood of the human to be treated at least during certain time intervals.

The term “effective amount” or “therapeutically effective amount” of a combination of therapeutic agents is an amount sufficient to provide an observable improvement over the baseline clinically observable signs and symptoms of the proliferative disease treated with the combination.

The term “synergistic effect” as used herein refers to action of two therapeutic agents producing an effect, for example, slowing the symptomatic progression of a cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively. Synergy may be further shown by calculating the synergy score of the combination according to methods known by one of ordinary skill. For this triple combination, the term “synergistic effect” as used herein refers to action of three therapeutic agents such as, for example, (a) an alpha-isoform specific PI3K inhibitor, (b) an mTOR inhibitor and (c) exemestane, producing an effect, for example, slowing the symptomatic progression of a cancer or symptoms thereof which is greater than the simple addition of the effects of each drug administered by themselves or greater than either dual therapy.

The term “subject” or “patient” as used herein includes animals, which are capable of suffering from or afflicted with a proliferative disease. Examples of subjects include mammals, e.g., humans, dogs, cows, horses, pigs, sheep, goats, cats, mice, rabbits rats and transgenic non-human animals. In the preferred embodiment, the subject is a human, e.g., a human suffering from, at risk of suffering from, or potentially capable of suffering from a proliferative disease.

The term “about” or “approximately” shall have the meaning of within 10%, more preferably within 5%, of a given value or range.

The present invention relates to a pharmaceutical combination comprising: (a) an alpha-isoform specific phosphatidylinositol-3-kinase (PI3K) inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor and (c) exemestane or any pharmaceutically acceptable salt thereof, particularly for separate, simultaneous or sequential use for the treatment or prevention of a proliferative disease (especially a cancer).

The alpha-isoform specific phosphatidylinositol -3-kinase (PI3K) inhibitor suitable for the present invention is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof.

WO2010/029082 describes specific 2-carboxamide cycloamino urea derivatives which have been found to be highly selective for the alpha isoform of phosphatidylinositol-3-kinase. The compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (hereinafter “COMPOUND A”) has the chemical structure of formula (I)

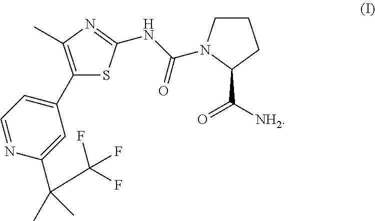 [SMILES: Cc1nc(NC(=O)N2CCC[C@H]2C(N)=O)sc1-c1ccnc(C(C)(C)C(F)(F)F)c1]

The compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), its salts, its utility as an alpha-isoform specific PI3K inhibitor and synthesis of the compound are described in WO2010/029082, which is hereby incorporated by reference in its entirety, for instance in Example 15.

COMPOUND A may be present in the form of the free base or any pharmaceutically acceptable salt thereof. Such salt(s), can be present alone or in mixture with free compound of the formula (I) and is preferably a pharmaceutically acceptable salt. As known in the field, such salts are formed, for example, as acid addition salts, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For therapeutic use, only pharmaceutically acceptable salts or free compounds are employed (where applicable in the form of pharmaceutical preparations).

Preferably, COMPOUND A is in the form of its free base.

Mammalian target of rapamycin (mTOR) inhibitors are known in the art. mTOR inhibitors particularly suitable for use in the present invention include, but is not limited to, compounds, proteins or antibodies which target/inhibit the activity/function of members of the mTOR kinase family, e.g., RAD, rapamycin (sirolimus which is also known by the name RAPAMUNE) and derivatives/analogs thereof such as everolimus (RAD001, Novartis) or compounds that inhibit the kinase activity of mTOR by directly binding to the ATP-binding cleft of the enzyme. Everolimus (RAD001) is also known by the name CERTICAN® or AFINITOR®.

Suitable mTOR inhibitors include e.g.:

I. Rapamycin which is an immunosuppressive lactam macrolide that is produced by Streptomyces hygroscopicus.

II. Rapamycin derivatives such as:

a. substituted rapamycin e.g. a 40-O-substituted rapamycin e.g. as described in U.S. Pat. No. 5,258,389, WO 94/09010, WO 92/05179, U.S. Pat. No. 5,118,677, U.S. Pat. No. 5,118,678, U.S. Pat. No. 5,100,883, U.S. Pat. No. 5,151,413, U.S. Pat. No. 5,120,842, WO 93/11130, WO 94/02136, WO 94/02485 and WO 95/14023 all of which are incorporated herein by reference;

b. a 16-O-substituted rapamycin e.g. as disclosed in WO 94/02136, WO 95/16691 and WO 96/41807, the contents of which are incorporated herein by reference;

c. a 32-hydrogenated rapamycin e.g. as described in WO 96/41807 and U.S. Pat. No. 5,256,790, incorporated herein by reference.

d. Preferred rapamycin derivatives are compounds of formula (II)

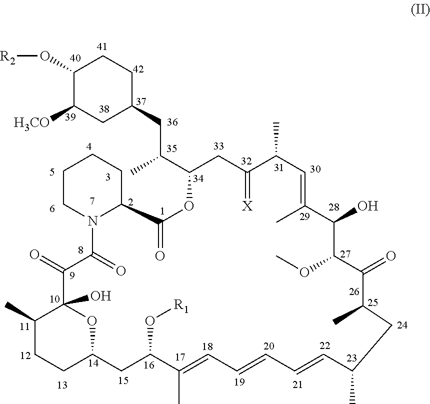

wherein
R₁ is CH₃ or C₃₋₆alkynyl,
R₂ is H or —CH₂—CH₂—OH, 3-hydroxy-2-(hydroxymethyl)-2-methyl-propanoyl or tetrazolyl, and X is ═O, (H,H) or (H,OH)
provided that R₂ is other than H when X is ═O and R₁ is CH₃,
or a prodrug thereof when R₂ is —CH₂—CH₂—OH, e.g. a physiologically hydrolysable ether thereof.

Compounds of formula (II) are disclosed e.g. in International PCT Applications WO94/09010, WO95/16691 or WO 96/41807, which are incorporated herein by reference. They may be prepared as disclosed or by analogy to the procedures described in these references.

Preferred compounds are 32-deoxorapamycin, 16-pent-2-ynyloxy-32-deoxorapamycin, 16-pent-2-ynyloxy-32(S)-dihydro-rapamycin, 16-pent-2-ynyloxy-32(S)-dihydro-40-O-(2-hydroxyethyl)-rapamycin and, more preferably, 40-0-(2-hydroxyethyl)-rapamycin, disclosed as Example 8 in International PCT Application WO94/09010.

Particularly preferred rapamycin derivative compounds of formula (II) are 40-O-(2-hydroxyethyl)-rapamycin, 40-[3-hydroxy-2-(hydroxymethyl)-2-methylpropanoate]-rapamycin (also called temsirolimus or CCI779), 40-epi-(tetrazolyl)-rapamycin (also called zotarolimus or ABT578), 32-deoxorapamycin, 16-pent-2-ynyloxy-32(S)-dihydro rapamycin, or TAFA-93.

e. Rapamycin derivatives also include so-called rapalogs, e.g. as disclosed in International PCT Applications WO98/02441 and WO01/14387, e.g. AP23573, AP23464, or AP23841.

Rapamycin and derivatives thereof have, on the basis of observed activity, e.g. binding to macrophilin-12 (also known as FK-506 binding protein or FKBP-12), e.g. as described in International PCT Applications WO94/09010, WO95/16691 or WO96/41807, been found to be useful e.g. as immuno-suppressant, e.g. in the treatment of acute allograft rejection.

III. Ascomycin, which is an ethyl analog of FK506.

IV. AZD08055 (AstraZeneca) and OSI-027 (OSI Pharmaceuticals), which are compounds that inhibit the kinase activity of mTOR by directly binding to the ATP-binding cleft of the enzyme.

V. SAR543, deforolimus (AP23573/MK-8669, Ariad/Merck & Co.), AP23841 (Ariad), KU-0063794 (AstraZeneca/Kudos), INK-128 (Intellikine), EX2044, EX3855, EX7518, WYE-125132 (Wyeth), XL765 (Exelisis), NV-128 (Novogen), WYE-125132 (Wyeth), EM101/LY303511 (Emiliem).

A preferred mTOR inhibitor for the present invention is everolimus (RAD001). Everolimus has the chemical name ((1R,9S,12S,15R,16E,18R,19R,21R,23S,24E,26E,28E,30S,32S,35R)-1,18-dihydroxy-12-{(1R)-2-[(1S,3R,4R)-4-(2-hydroxyethoxy)-3-methoxycyclohexyl]-1-methylethyl1-19,30-dimethoxy-15,17,21,23,29,35-hexamethyl-11,36-dioxa-4-aza-tricyclo[30.3.1.04,9] hexatriaconta-16,24, 26,28-tetraene-2,3,10,14,20-pentaone.) Everolimus and analogues are described in U.S. Pat. No. 5,665,772, at column 1, line 39 to column 3, line 11.

The specific aromatase inhibitor useful in the present invention is exemestane. Exemestane is chemically described as 6-methylenandrosta-1,4-diene-3,17-dione and has the following chemical structure:

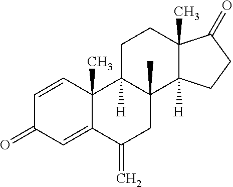

Exemestane is described in U.S. Pat. No. 4,808,616, which is hereby incorporated by reference in its entirety, and can be prepared and formulated as disclosed therein. Further, exemestane can be administered, e.g., in the form as it is marketed, e.g. under the trademark AROMASIN® (Pfizer Inc.).

The structure of the active ingredients identified by code numbers., generic or trade names may be taken from the actual edition of the standard compendium “The Merck Index” or from databases, e.g., Patents International (e.g, IMS World Publications). The corresponding content thereof is hereby incorporated by reference.

A “pharmaceutically acceptable salt” of the mTOR inhibitor or the aromatase inhibitor exemestane, as used herein, unless otherwise indicated, includes salts of acidic and basic groups which may be present in the compounds of the present invention. Such salts can be prepared, e.g., by separately reacting the base or acid functions with a suitable organic or inorganic acid or base, respectively. Suitable salts of the compound include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2 hydroxyethanesulfonate, lactate, maleate, methanesulfonate, nicotinate, 2 naphth-alenesulfonate, oxalate, pamoate, pectinate, persulfate, 3 phenylproionate, picrate, pivalate, propionate, succinate, sulfate, tartrate, thiocyanate, p toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others.

Hereinafter, the triple combination comprising (a) an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor and (c) exemestane or any pharmaceutically acceptable salt thereof, will be referred to as a COMBINATION OF THE INVENTION.

In one embodiment, the COMBINATION OF THE INVENTION comprises (a) an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor selected from selected from RAD, rapamycin (sirolimus) and derivatives/analogs thereof (such as everolimus, temsirolimus, and zotarolimus), SAR543, ascomycin, deforolimus, AP23841, KU-0063794, INK-128, EX2044, EX3855, EX7518, AZD08055, OSI-027, WYE-125132, XL765, NV-128, WYE-125132, EM101/LY303511 or any pharmaceutically acceptable salts thereof and (c) exemestane or any pharmaceutically acceptable salt thereof.

In a preferred embodiment, the COMBINATION OF THE INVENTION comprises (a) an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor everolimus or any pharmaceutically acceptable salt thereof and (c) exemestane or any pharmaceutically acceptable salt thereof.

Unless otherwise specified, or clearly indicated by the text, or not applicable, reference to therapeutic agents useful in the COMBINATION OF THE INVENTION includes both the free base of the compounds, and all pharmaceutically acceptable salts of the compounds.

The present invention particularly pertains to a COMBINATION OF THE INVENTION useful for separate, simultaneous or sequential administration to a subject in need thereof for treating or preventing a proliferative disease (particularly a cancer).

The present invention particularly pertains to a COMBINATION OF THE INVENTION useful for treating or preventing a proliferative disease in a subject in need thereof. In one embodiment of the present invention, the COMBINATION OF THE INVENTION is used for the treatment or prevention of a proliferative disease comprising administering to the subject a pharmaceutical combination comprising an effective amount of an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, an effective amount of an mTOR inhibitor (especially everolimus) and an effective amount of an aromatase inhibitor exemestane or any pharmaceutically acceptable salt thereof. Preferably, these therapeutic agents are administered at therapeutically effective dosages which, when combined provide a beneficial effect. The administration may be separate, simultaneous or sequential.

The COMBINATION OF THE INVENTION is particularly useful for the treatment or prevention of a proliferative disease in a subject in need thereof. Examples of proliferative diseases for treatment or prevention with the COMBINATION OF THE INVENTION include, but are not limited to, cancer, graft-versus-host disease, restenosis, hamartoma syndromes (e.g., tuberous sclerosis or Cowden Syndrome), encephalomyelitis, insulin-dependent diabetes mellitus, lupus, dermatomyositis, arthritis, rheumatic diseases, scleroderma, pulmonary fibrosis, renal fibrosis, cystic fibrosis, pulmonary hypertension, immunomodulation, multiple sclerosis, VHL syndrome, Carney complex, Familial adenonamtous polyposis, juvenile polyposis syndrome, Birt-Hogg-Duke syndrome, hypertrophic cardiomyopathy, Wolf-Parkinson-White syndrome, neurodegenerative diseases (e.g, Parkinson's, Huntington's, Alzheimer's and dementias caused by tau mutations, spinocerebellar ataxia type 3, motor neuron disease caused by SOD1 mutations, neuronal ceroid lipofucinoses/Batten disease, etc.), wet and dry macular degeneration, muscle wasting (atrophy, cachexia) and myopathies (e.g., Danon's disease), bacterial and viral infections (e.g., including M. tuberculosis, group A streptococcus, HSV type I, HIV infection), neurofibromatosis, and Peutz-Jeghers Syndrome.

Preferably, the proliferative disease is a cancer. The term “cancer” is used herein to mean a broad spectrum of benign and malignant tumors, including all solid tumors and hematological malignancies. Examples of such tumors include but are not limited to benign or malignant tumors of the brain, kidney (e.g, renal cell carcinoma), liver, adrenal gland, bladder, breast, stomach, gastric, gastrointestine, ovaries, colon, rectum, prostate, pancreas, lung (e.g,. small cell lung cancer and non-small cell lung cancer), uterus, vagina, thyroid, neuroendocrine (e.g, pancreatic neuroendocrine tumor), sarcoma, glioblastomas, multiple myeloma, colorectal adenoma, neck and head, endometrial, melanoma, an epidermal hyperproliferation, psoriasis, prostate hyperplasia, a neoplasia, a neoplasia of epithelial character, lymphomas (e.g., non-Hodgkin lymphoma and Hodgkin lymphoma), a mammary carcinoma, a leukemia (e.g., acute myelogenous leukemia, chronic myelogenous leukemia, lymphocytic leukemia, and myeloid leukemia), and combinations thereof.

The COMBINATION OF THE INVENTION inhibits the growth of solid tumors, but also liquid tumors. In a further embodiment of the present invention, the cancer is a solid tumor. The term “solid tumor” especially means breast cancer, ovarian cancer, colon cancer, rectal cancer, gastrointestinal cancer, cervix cancer, lung cancer (e.g., small-cell lung cancer and non-small cell lung cancer), kidney cancer (e.g, renal cell carcinoma), neuroendocrine tumor (e.g., pancreatic neuroendocrine tumor), melanoma, head and neck cancer, bladder cancer, and prostate cancer. Further, depending on the tumor type and particular combination used, a decrease of the tumor volume can be obtained. The COMBINATION OF THE INVENTION disclosed herein is also suited to prevent the metastatic spread of tumors and the growth or development of micrometastases. In a preferred embodiment, the COMBINATION OF THE INVENTION disclosed herein is used of the treatment of a cancer.

The COMBINATION OF THE INVENTION disclosed herein is suitable for the treatment of poor prognosis patients, especially such poor prognosis patients having a cancer which is resistant to treatment employing an mTOR inhibitor or an aromatase inhibitor as a sole therapeutic agent (e.g., a cancer of such patients who initially had responded to treatment with an mTOR inhibitor or or aromatase inhibitor and then relapsed), or such poor prognosis patients having a cancer which is resistant to treatment employing an mTOR inhibitor and an aromatase inhibitor as therapeutic agents (e.g., a cancer of such patients who initially had responded to treatment with an mTOR inhibitor and an aromatase inhibitor and then relapsed). This cancer may have acquired resistance during prior treatment with one or more mTOR inhibitors, e.g., one of those listed above and incorporated herein by reference, e.g, everolimus or any pharmaceutically acceptable salt thereof. This cancer may have acquired resistance during prior treatment with one or more aromatase inhibitors, e.g., exemestane, letrozole or anastrozole. Thus, in one embodiment, the cancer is resistant to treatment employing an mTOR inhibitor as a sole therapeutic agent.

In one preferred embodiment, the cancer is breast cancer, pancreatic neuroendocrine tumor or renal cell carcinoma. In a further preferred embodiment, the cancer is breast cancer. In a further preferred embodiment, the cancer is a hormone-receptor positive breast cancer or estrogen-receptor positive breast cancer.

Further, the COMBINATION OF THE INVENTION is particularly useful for the treatment or prevention of a proliferative disease (particularly a cancer) associated with or having a molecular link to a dysregulation of the mTOR kinase and/or having an overexpression or amplification of PI3K alpha, somatic mutation of PIK3CA or germline mutations or somatic mutation of PTEN or mutations and translocation of p85α that serve to up-regulate the p85-p110 complex.

In one embodiment, the present invention relates to the COMBINATION OF THE INVENTION for use in the treatment or prevention of a proliferative disease, preferably a cancer.

In a further embodiment, the present invention relates to the COMBINATION OF THE INVENTION for use in the treatment or prevention of a breast cancer.

In a preferred embodiment, the present invention relates to a pharmaceutical combination comprising (a) an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor everolimus or any pharmaceutically acceptable salt thereof, and (c) exemestane or any pharmaceutically acceptable salt thereof for use in the treatment or prevention of a hormone-receptor positive breast cancer.

In a further embodiment, the present invention relates to a COMBINATION OF THE INVENTION for use in the prevention of the metastatic spread of tumors or the growth or development of micrometastases in a subject in need thereof.

In one embodiment, the present invention relates to a method for treating or preventing a proliferative disease (preferably a cancer), in a subject in need thereof comprising administering to said subject a therapeutically effective amount of a COMBINATION OF THE INVENTION. In each embodiment, COMBINATION OF THE INVENTION is preferably administered in a quantity that is jointly therapeutically effective for the treatment of said proliferative disease in a patient suffering from said proliferative disease.

In a further embodiment, the present invention relates to a method for treating or preventing a breast cancer, in a subject in need thereof comprising administering to said subject a jointly therapeutically effective amount of a COMBINATION OF THE INVENTION.

In a preferred embodiment, the present invention relates to a method for treating or preventing a hormone-receptor positive breast cancer in a subject in need thereof comprising administering to said subject a jointly therapeutically effective amount of (a) an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor everolimus or any pharmaceutically acceptable salt thereof, and (c) exemestane or any pharmaceutically acceptable salt thereof.

In a further embodiment, the present invention relates to a method for preventing the metastatic spread of tumors or the growth or development of micrometastases in a subject in need thereof comprising comprising simultaneously, separately or sequentially administering to said subject a jointly therapeutically effective amount of a COMBINATION OF THE INVENTION.

In one embodiment, the present invention relates to the use of a COMBINATION OF THE INVENTION for the preparation of a pharmaceutical composition or medicament for the treatment or prevention of a proliferative disease (preferably a cancer).

In a further embodiment, the present invention relates to the use of a COMBINATION OF THE INVENTION for the preparation of a pharmaceutical composition or medicament for the treatment or prevention of a breast cancer.

In a preferred embodiment, the present invention relates to the use of a pharmaceutical combination comprising (a) an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor everolimus or any pharmaceutically acceptable salt thereof and (c) exemestane or any pharmaceutically acceptable salt thereof for the preparation of a pharmaceutical composition or medicament for the treatment or prevention of a hormone-receptor positive breast cancer.

In a further embodiment, the present invention relates to the use of a COMBINATION OF THE INVENTION for the preparation of a pharmaceutical composition or medicament for the prevention of the metastatic spread of tumors or the growth or development of micrometastases.

In one embodiment, the present invention relates to the use of the COMBINATION OF THE INVENTION for the treatment or prevention of a proliferative disease (preferably a cancer).

In a further embodiment, the present invention relates to the use of a COMBINATION OF THE INVENTION for the treatment or prevention of a breast cancer.

In a preferred embodiment, the present invention relates to the use of a pharmaceutical combination comprising (a) an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor everolimus or any pharmaceutically acceptable salt thereof and (c) exemestane or any pharmaceutically acceptable salt thereof for the treatment or prevention of a hormone-receptor positive breast cancer.

In a further embodiment, the present invention relates to the use of a COMBINATION OF THE INVENTION for the prevention of the metastatic spread of tumors or the growth or development of micrometastases.

The nature of any cancer is multifactorial. Under certain circumstances, drugs with different mechanisms of action may be combined. However, just considering any combination of therapeutic agents having different mode of action does not necessarily lead to combinations with advantageous effects.

The administration of a COMBINATION OF THE INVENTION may result not only in a beneficial effect, e.g. a synergistic therapeutic effect, e.g, with regard to anti-proliferative activity, e.g. with regard to alleviating, delaying progression of or inhibiting the symptoms, but also in further surprising beneficial effects, e.g. fewer side-effects, more durable response, an improved quality of life or a decreased morbidity, compared with a monotherapy applying only one of the therapeutic agents used in the COMBINATION OF THE INVENTION.

A further benefit is that lower doses of the therapeutic agents of the COMBINATION OF THE INVENTION can be used, for example, that the dosages need not only often be smaller, but are also applied less frequently, or can be used in order to diminish the incidence of side-effects observed with one of the therapeutic agents alone. This is in accordance with the desires and requirements of the patients to be treated.

It can be shown by established test models that a COMBINATION OF THE INVENTION results in the beneficial effects described herein before. The person skilled in the art is fully enabled to select a relevant test model to prove such beneficial effects. The pharmacological activity of a COMBINATION OF THE INVENTION may, for example, be demonstrated in a clinical study or in an in vivo or in vitro test procedure as essentially described hereinafter.

Suitable clinical studies are in particular, for example, open label, dose-escalation or safety and efficacy studies in patients with a proliferative disease (especially a cancer). Such studies prove in particular the synergism or improved antiproliferative effect of the therapeutic agents of the COMBINATION OF THE INVENTION. The beneficial effects on one or more proliferative diseases may be determined directly through the results of these studies which are known as such to a person skilled in the art. Such studies may be, in particular, be suitable to compare the effects of a monotherapy using either therapeutic agent or a dual therapy using two therapeutic agents and a COMBINATION OF THE INVENTION. In one embodiment, the dose of the alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof, is escalated until the Maximum Tolerated Dosage is reached, and the mTOR inhibitor and the aromatase inhibitor exemestane are administered with a fixed dose. Alternatively, the dose of the alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof, and the mTOR inhibitor is escalated until the Maximum Tolerated Dosage is reached, and the aromatase inhibitor exemestane is administered with a fixed dose. Alternatively, the alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof, may be administered in a fixed dose and the dose of the mTOR inhibitor and/or the aromatase inhibitor exemestane may be escalated. Such studies may be, in particular, be suitable to compare the effects of a monotherapy or dual therapy to a triple pharmaceutical combination therapy of the present invention. Each patient may receive doses of the PI3K inhibitor either daily or intermittently. The efficacy of the treatment may be determined in such studies, e.g., after 8, 16, 24, or 32 weeks by evaluation of tumor size or progression and/or evaluation of symptom scores every 8 weeks.

Determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients may render impractical the use of this form of testing as a primary model for synergy. However, the observation of synergy in one species can be predictive of the effect in other species and animal models exist, as described herein, to measure a synergistic effect and the results of such studies can also be used to predict effective dose ratio ranges and the absolute doses and plasma concentrations required in other species by the application of pharmacokinetic/pharmacodynamic methods. Established correlations between tumor models and effects seen in man suggest that synergy in animals may be demonstrated, for example, by xenograft models or in appropriate cell lines.

The alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) is generally administered orally at a dose in the range from about 30 mg to about 450 mg, or about 100 mg to about 400 mg, or about 300 mg to about 400 mg, or about 250 mg to about 350 mg per day in a human adult. Preferably, COMPOUND A is administered orally at a dose of about 250 mg to about 350 mg per day in a human adult. The daily dose can be administered on a qd or bid schedule.

The mTOR inhibitor everolimus may be administered orally to a human in a daily dosage range of 0.5 to 1000 mg; preferably in the range of 0.5 mg to 15 mg; most preferably in the range of 0.5 mg to 10 mg.

The aromatase inhibitor exemestane may be administered orally to a human in a dosage range varying from 5 to 200 mg/day, preferably from 10 to 25 mg/day, or parenterally from 50 to 500 mg/day, preferably from 100 to 250 mg/day. If the therapeutic agent shall be administered in a separate pharmaceutical composition, it can be administered in the form disclosed in GB 2,177,700.

It is understood that each therapeutic agent may be conveniently administered, for example, in one individual dosage unit or divided into multiple dosage units. It is further understood that that each therapeutic agent may be conveniently administered in doses once daily or doses up to four times a day.

In one embodiment, the present invention relates to a pharmaceutical composition or combined preparation comprising a quantity, which is jointly therapeutically effective against a proliferative disease (particularly a cancer), of the COMBINATION OF THE INVENTION, and optionally at least one pharmaceutically acceptable carrier. In this pharmaceutical composition, the therapeutic agents (i.e., alpha-isoform specific PI3K inhibitor and/or the mTOR inhibitor and/or the aromatase inhibitor exemestane) can be administered in a single formulation or unit dosage form, administered concurrently but separately, or administered sequentially by any suitable route. Preferably, the alpha-isoform specific PI3K inhibitor, the mTOR inhibitor and the aromatase inhibitor exemestane are administered concurrently but separately.

A therapeutically effective amount of the therapeutic agents of the COMBINATION OF THE INVENTION may be administered simultaneously or sequentially and in any order, and the components may be administered separately or as a fixed combination. For example, the method of treatment or prevention of a cancer, according to the invention may comprise (i) administration of the first therapeutic agent in free or pharmaceutically acceptable salt form and (ii) administration of the second therapeutic agent in free or pharmaceutically acceptable salt form, and (iii) administration of the third therapeutic agent in free or pharmaceutically acceptable salt form, separately, simultaneously or sequentially in any order, in jointly therapeutically effective amounts (preferably in synergistically effective amounts). The individual therapeutic agents of the COMBINATION OF THE INVENTION can be administered separately at different times during the course of therapy or concurrently in divided or single combination forms. The invention is therefore to be understood as embracing all such regimens of simultaneous or alternating treatment and the term “administering” is to be interpreted accordingly. Preferably, the alpha-isoform specific PI3K inhibitor, the mTOR inhibitor and the aromatase inhibitor exemestane are administered separately.

The effective dosage of each therapeutic agent employed in the COMBINATION OF THE INVENTION may vary depending on the particular compound or pharmaceutical composition employed, the mode of administration, the condition being treated, and the severity of the condition being treated. Thus, the dosage regimen of the COMBINATION OF THE INVENTION is selected in accordance with a variety of factors including the route of administration and the renal and hepatic function of the patient. A clinician or physician of ordinary skill can readily determine and prescribe the effective amount of the single therapeutic agents required to alleviate, counter or arrest the progress of the condition.

The effective dosage of each of the therapeutic agents used in the COMBINATION OF THE INVENTION may require more frequent administration of one of the therapeutic agent(s) as compared to the other therapeutic agent(s) in the combination. Therefore, to permit appropriate dosing, packaged pharmaceutical products may contain one or more dosage forms that contain the combination of therapeutic agents, and one or more dosage forms that contain one of the combination of therapeutic agents, but not the other therapeutic agent(s) of the combination.

When any of the therapeutic agents employed in the COMBINATION OF THE INVENTION, are applied in the form as marketed as single drugs, their dosage and mode of administration can be in accordance with the information provided on the package insert of the respective marketed drug, if not mentioned herein otherwise.

The optimum ratios, individual and combined dosages, and concentrations of the therapeutic agents (a), (b) and (c) employed in the COMBINATION OF THE INVENTION that yield efficacy without toxicity are based on the kinetics of the therapeutic agents' availability to target sites, and are determined using methods known to those of skill in the art.

The optimal dosage of each therapeutic agent for treatment or prevention of a cancer can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art.

The amount of each therapeutic agent of the COMBINATION OF THE INVENTION that may be combined with the carrier materials to produce a single dosage form will vary depending upon the individual treated and the particular mode of administration. In some embodiments the unit dosage forms containing the combination of agents as described herein will contain the amounts of each therapeutic agent of the combination that are typically administered when the therapeutic agents are administered alone.

Frequency of dosage may vary depending on the compound used and the particular condition to be treated or prevented. Patients may generally be monitored for therapeutic effectiveness using assays suitable for the condition being treated or prevented, which will be familiar to those of ordinary skill in the art.

The pharmaceutical composition according to the invention can be prepared in a manner known per se and are those suitable for enteral, such as oral or rectal, and parenteral administration to mammals (warm-blooded animals), including man. Alternatively, when the agents are administered separately, one can be an enteral formulation and the other can be administered parenterally.

Preferably, the pharmaceutical composition comprising the alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof is suitable for enteral administration.

The novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, sachets and furthermore ampoules. If not indicated otherwise, these are prepared in a manner known per se, for example by means of conventional mixing, granulating, sugar-coating, dissolving or lyophilizing processes. It will be appreciated that the unit content of one of the therapeutic agents contained in an individual dose of each dosage form need not in itself constitute an effective amount since the necessary effective amount can be reached by administration of a plurality of dosage units.

In preparing the compositions for oral dosage form, any of the usual pharmaceutically acceptable carriers may be employed, such as, for example, water, glycols, oils, alcohols, flavoring agents, preservatives, coloring agents; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

One of ordinary skill in the art may select one or more of the aforementioned carriers with respect to the particular desired properties of the dosage form by routine experimentation and without any undue burden. The amount of each carriers used may vary within ranges conventional in the art. The following references which are all hereby incorporated by reference disclose techniques and excipients used to formulate oral dosage forms. See The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2003).

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

In a further embodiment, the present invention relates to a combined preparation comprising (a) one or more dosage units of an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof and (b) one or more dosage units of mTOR inhibitor, and (c) one or more dosage units of exemestane or any pharmaceutically acceptable salt thereof for use in the treatment or prevention of a proliferative disease (preferably a cancer).

In one embodiment, the present invention provides a commercial package comprising as active ingredients of COMBINATION OF THE INVENTION and instructions for simultaneous, separate or sequential administration of said combination to a patient in need thereof for use in the treatment or prevention of a proliferative disease (preferably a cancer).

In a further embodiment, the present invention provides a commercial package comprising as active ingredient an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and instructions for simultaneous, separate or sequential administration of said active ingredient with an mTOR inhibitor (particularly everolimus) and an aromatase inhibitor exemestane or any pharmaceutically acceptable salt thereof to a patient in need thereof for use in the treatment or prevention of a proliferative disease (preferably a cancer).

In a preferred embodiment, the present invention provides a commercial package comprising as active ingredient an alpha-isoform specific PI3K inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and instructions for simultaneous, separate or sequential administration of said active ingredient with an mTOR inhibitor everolimus or any pharmaceutically acceptable salt thereof and exemestane or any pharmaceutically acceptable salt thereof to a patient in need thereof for use in the treatment or prevention of a hormone-receptor positive breast cancer.

The following Examples illustrate the invention described above; they are not, however, intended to limit the scope of the invention in any way. The beneficial effects of the pharmaceutical combination of the present invention can also be determined by the clinical trial as described below or other test models known as such to the person skilled in the pertinent art.

### EXAMPLE 1

A clinical study using (a) an alpha-isoform specific phosphatidylinositol 3-kinase inhibitor COMPOUND A in combination with (b) an mTOR inhibitor everolimus and (c) an aromatase inhibitor exemestane for treatment of post-menopausal female patients with advanced hormone receptor-positive/HER2-negative breast cancer.

An open-label, multi-center Phase Ib dose-finding study and safety and efficacy clinical trial of the combination comprising (a) an alpha-isoform specific phosphatidylinositol 3-kinase inhibitor COMPOUND A in combination with (b) an mTOR inhibitor everolimus and (c) an aromatase inhibitor exemestane is conducted in post-menopausal female patients with advanced hormone receptor-positive/HER2-negative breast cancer.

In this study, the treatment dose-escalation phase is conducted in two steps: (a) first, the maximal tolerated dose (MTD) of the COMPOUND A and everolimus double combination is determined in patients with metastatic and/or recurrent solid tumors (“Dose Escalation Phase I”), and (b) second, after the MTD is determined for the COMPOUND A and everolimus double combination, the MTD is determined for the COMPOUND A, everolimus, and exemestane triple combination (“Dose Escalation Phase II”). Upon completion of Dose Escalation Phase II, two dose expansion arms are conducted to evaluate the safety and efficacy of the COMPOUND A, everolimus, and exemestane triple combination in post-menopausal women with a mTOR inhibitor-naive hormone-receptor positive/HER2-negative breast cancer (Expansion Arm A) and in post-menopausal women with a mTOR inhibitor-pretreated hormone-receptor positive/HER2-negative breast cancer (Expansion Arm B).

Approximately 15-25 patients are enrolled in Dose Escalation Phase I for the COMPOUND A and everolimus double combination. In Dose Escalation Phase I, patients consist of adult individuals (both female and males) with metastatic and/or recurrent solid tumors with an adequate performance status for whom no standard therapy exists.

After the MTD for the COMPOUND A and everolimus double combination is determined, Dose Escalation Phase II is begun only in post-menopausal women with advanced hormone-receptor positive/HER2-negative breast cancer. Approximately 5 to 15 patients are enrolled. Once the MTD for the triple combination is determined, approximately 40 post-menopausal women with advanced mTOR inhibitor-naïve hormone receptor positive/HER2 negative breast cancer are evaluated in Expansion Arm A and approximately 10 post-menopausal women with advanced hormone receptor positive/HER2 negative breast cancer who had been previously treated with an mTOR inhibitor are evaluated in in Expansion Arm B.

The eligibility of patients is determined during a screening period, which occurs within 1 to 28 days prior to treatment start, with exception of the hematology, full chemistry, coagulation, fasting plasma glucose and fasting C-peptide and insulin assessment that is done 1 to 7 days prior to start of study treatment and serum pregnancy test that is done within 3 days prior to start of study treatment. Eligible patients must provide a signed study Informed Consent Form prior to any screening procedure and be an adult ≥18 years of age that is able to comply with clinical study protocol requirements. The following general screening inclusion criteria is used for Dose Escalation Phase I, Dose Escalation Phase II, Expansion Arm A and Expansion Arm B:
1. Patient has tumor tissue available for the analysis of PI3K signaling.
2. Patient has an Eastern Cooperative Oncology Group Performance Status (ECOG PS) ≤2 that the investigator believes is stable at the time of screening
3. Patient has adequate bone marrow and organ function as defined by the following laboratory values:
   Absolute Neutrophil Count (ANC) ≥1.5×10⁹/L
   Platelets ≥100×10⁹/L
   Hemoglobin ≥90 g/L
   INR ≤2
   Serum creatinine ≤1.5×ULN
   Total serum bilirubin ≤2.0 mg/dL
   Alanine aminotransferase (AST) and aspartate aminotransferase (ALT)≤2.5×ULN (or ≤5×ULN if liver metastases are present)
   Fasting plasma glucose (FPG) ≤140 mg/dL or ≤7.8 mmol/L
   Cholesterol (fasting) ≤300 mg/dL OR ≤7.75 mmol/L AND fasting triglycerides ≤2.5×ULN. NOTE: In case one or both of these thresholds are exceeded, the patient can only be included after initiation of appropriate lipid lowering medication
4. Patient is able to swallow and retain oral medication
5. Patient has either measurable or non-measurable disease as per RECIST 1.1.
One additional inclusion criteria for Dose Escalation Phase I only includes:
6. Patient has a histologically/cytologically confirmed metastatic and/or recurrent solid tumor for whom no standard therapy exists
Additional inclusion criteria for Expansion Arm A and Expansion Arm B:
6. Patient has a histologically and/or cytologically confirmed diagnosis of breast cancer.
7. Patient has radiologic evidence of inoperable locally advanced, or metastatic breast cancer.
8. Patient has HER2-negative breast cancer (based on most recently analyzed biopsy) defined as a negative in situ hybridization by approved test or an IHC status of 0, 1+ or 2+ (if IHC 2+, a negative in situ hybridization test is required) by local laboratory testing
9. Patient has a known hormone receptor-positive status (either estrogen or progesterone) by local laboratory testing based on most recently analyzed biopsy
10. Patient is a postmenopausal woman. Postmenopausal status is defined either by:
   Prior bilateral oophorectomy (with or without hysterectomy)
   Age ≥60
   Age <60 and amenorrhea for ≥12 months (in the absence of chemotherapy, tamoxifen, toremifen, or ovarian suppression) and FSH and estradiol in the postmenopausal range (according to local laboratory ranges or if not available: serum FSH >40 mIU/mL and estradiol <20 pg/mL).
For women with therapy-induced amenorrhea, oophorectomy or serial measurements of FSH and/or estradiol are needed to ensure postmenopausal status. Note: Ovarian radiation or treatment with a luteinizing hormone-releasing hormone (LH-RH) agonist (goserelin acetate or leuprolide acetate) is not permitted for induction of ovarian suppression
11. Patient whose disease is refractory to previous letrozole or anastrozole, defined as recurrence during or within 12 months after the end of adjuvant treatment or progression during or within 1 month after the end of treatment for advanced disease.
12. Patient must have received up to two prior chemotherapy regimens for locally advanced or metastatic disease. NOTE: Adjuvant/neoadjuvant therapy will be counted as one prior line of therapy for metastatic/recurrent disease if the patient had a progression/recurrence within 6 months after completion of the therapy.
One additional inclusion criteria for the Expansion Arm A includes:

13. Patient has received and progressed to a prior mTOR inhibitor treatment. Patient who had discontinued prior mTOR inhibitor treatment due to non-tolerable toxicities is not eligible.
Patients must meet all screening inclusion criteria to be eligible.

The following general screening exclusion criteria is used for Dose Escalation Phase I, Dose Escalation Phase II, Expansion Arm A and Expansion Arm B:
1. Patient has received previous treatment with a PI3K and/or AKT and/or mTOR inhibitor (e.g. sirolimus, temsirolimus, deforolimus). Note: Prior mTOR inhibitor treatment is allowed only in the mTOR inhibitor-pretreated patients' cohort (Expansion Arm A).
2. Known intolerance or hypersensitivity to Everolimus or other rapamycin analogs (e.g. sirolimus, temsirolimus)
3. Patient with primary central nervous system (CNS) tumor or CNS tumor involvement. However, patient with solid tumors that are metastatic to CNS may participate in this study if the patient is:
   a. 4 weeks from prior therapy completion (including radiation and/or surgery) to starting the study treatment, and
   b. Clinically stable with respect to the CNS tumor at the time of screening, and
   c. Not receiving steroid therapy
4. Patient with diabetes mellitus, or documented steroid-induced diabetes mellitus
5. Patient has a history of another malignancy within 2 years prior to starting study treatment, except for cured basal cell carcinoma of the skin or excised carcinoma in situ of the cervix.
6. Patient who has not recovered to grade 1 or better (except alopecia) from related side effects of any prior antineoplastic therapy
7. Patient who has had systemic therapy within 4 weeks (6 weeks for nitrosoureas or mitomycin C) prior to study entry.
8. Patient who has received radiotherapy 4 weeks prior to starting study drugs, with exception of palliative radiotherapy 2 weeks prior to starting study drugs), who has not recovered from side effects of such therapy to baseline or Grade 1 and/or from whom 30% of the bone marrow was irradiated.
9. Patient who has undergone major surgery 4 weeks prior to starting study treatment or who has not recovered from side effects of such procedure.
10. Patient has a clinically significant cardiac disease or impaired cardiac function, such as:
   a. Congestive heart failure (CHF) requiring treatment (New York Heart Association (NYHA) Grade ≥2), left ventricular ejection fraction (LVEF) <50% as determined by multi-gated acquisition (MUGA) scan or echocardiogram (ECHO),
   b. History or current evidence of clinically significant cardiac arrhythmias, atrial fibrillation and/or conduction abnormality, e.g. congenital long QT syndrome, high-grade/complete AV-blockage
   c. Acute coronary syndromes (including myocardial infarction, unstable angina, coronary artery bypass graft (CABG), coronary angioplasty, or stenting), <3 months prior to screening
   d. QT interval adjusted according to Fredericia (QTcF) >480 msec on screening ECG
11. Patient who has any severe and/or uncontrolled medical conditions such as:
   a. active or uncontrolled severe infection,
   b. liver disease such as cirrhosis, decompensated liver disease, and chronic hepatitis (i.e. quantifiable Hepatitis B Virus (HBV)-DNA and/or positive surface antigen of Hepatitis B virus (HbsAg), quantifiable HCV-RNA),
   c. known severely impaired lung function (spirometry and DLCO (diffusing capacity of the lung for carbon monoxide) 50% or less of normal and O₂ saturation 88% or less at rest on room air),
   d. active, bleeding diathesis;
   e. uncontrolled arterial hypertension defined by blood pressure >140/100 mm Hg at rest (average of 3 consecutive readings 5 min apart)
   f. Chronic treatment with corticosteroids or other immunosuppressive agents
12. Patient who is currently receiving medication with a known risk of prolonging the QT interval or inducing Torsades de Pointes (TdP) and the treatment cannot either be discontinued or switched to a different medication prior to starting study drug treatment.
13. Patient who has participated in a prior investigational study within 30 days prior to enrollment.
14. Patient who is currently receiving treatment with drugs known to be moderate or strong inhibitors or inducers of isoenzymes CYP34A or CYP2C8. The patient must have discontinued moderate and strong inducers of both enzymes for at least one week and must have discontinued strong and moderate inhibitors before the start of treatment. Switching to a different medication prior to start of treatment is allowed.
15. Patient with impaired gastrointestinal (GI) function or GI disease that may significantly alter the absorption of oral COMPOUND A, everolimus, exemestane (e.g. ulcerative disease, uncontrolled nausea, vomiting, diarrhea, malabsorption syndrome, or small bowel resection).
16. Patient with known positive serology for human immunodeficiency virus (HIV).
17. Patients who have received live attenuated vaccines within 1 week of start of study drug and during the study. Patient should also avoid close contact with others who have received live attenuated vaccines (e.g., intranasal influenza, measles, mumps, rubella, oral polio, BCG, yellow fever, varicella and TY21a typhoid vaccines).
18. Pregnant or nursing (lactating) woman, where pregnancy is defined as the state of a female after conception and until the termination of gestation, confirmed by a positive hCG laboratory test (>5 mIU/mL).
19. Patient who does not apply highly effective contraception during the study and through the duration as defined below after the final dose of study treatment:
   Sexually active males should use a condom during intercourse while taking drug and for 8 weeks after the final dose of study treatment and should not father a child in this period. A condom is required to be used also by vasectomized men in order to prevent delivery of the drug via seminal fluid.
   Women of child-bearing potential (defined as all women physiologically capable of becoming pregnant), unless they are using highly effective methods of contraception during dosing and 8 weeks after the final dose of study treatment. Highly effective contraception methods include:
   Total abstinence (when this is in line with the preferred and usual lifestyle of the subject. Periodic abstinence (e.g., calendar, ovulation, symptothermal, post-ovulation methods) and withdrawal are not acceptable methods of contraception
   Female sterilization (have had surgical bilateral oophorectomy with or without hysterectomy) or tubal ligation at least six weeks before taking study treatment. In case of oophorectomy alone, only when the reproductive status of the woman has been confirmed by follow up hormone level assessment
   Male partner sterilization (at least 6 months prior to screening). For female subjects on the study the vasectomized male partner should be the sole partner for that subject.
   Combination of the following methods:
      1. Placement of an intrauterine device (IUD) or intrauterine system (IUS)
      2. Barrier methods of contraception: Condom or Occlusive cap (diaphragm or cervical/vault caps) with spermicidal foam/gel/film/cream/vaginal suppository

Post-menopausal women are allowed to participate in this study. Women are considered post-menopausal and not of child bearing potential if they are:
Aged ≥60;
or aged <60 and have had 12 months of natural (spontaneous, in the absence of chemotherapy, tamoxifen, toremifen, or ovarian suppression) amenorrhea with and FSH and estradiol in the postmenopausal range (serum FSH >40 mIU/mL and estradiol <20 pg/mL or according to the postmenopausal range definition for the local laboratory involved)
or have had surgical bilateral oophorectomy (with or without hysterectomy)
For women with therapy-induced amenorrhea, oophorectomy or serial measurements of follicle stimulating hormone (FSH) and/or estradiol are needed to ensure postmenopausal status. NOTE: Ovarian radiation or treatment with a luteinizing hormone-releasing hormone agonist (goserelin acetate or leuprolide acetate) is not permitted for induction of ovarian suppression.

One additional exclusion criteria for the Expansion Arm B includes:
20. Patient who has discontinued prior mTOR inhibitor therapy due to non-tolerable toxicity

Patients must not meet any of the screening exclusion criteria to be eligible for the study.

Patients participating in the dose escalation part of the study are not permitted to be enrolled in the dose expansion part of the study.

During screening, patients are evaluated for patient history, IRT registration, physical examination (including ECOG performance status, height, weight, physical examination, and vital signs), laboratory assessments (including hematology, chemistry (full panel), fasting lipid panel, coagulation, fasting plasma glucose, fasting C-peptide, insulin, HBA1c, lipase, urinalysis, HBV/HCV screening, pregnancy), imaging (including tumor evaluation, 12-lead ECG, ophthalmic evaluation, cardiac imaging, pulmonary function tests), and safety (including adverse events, surgical/medical procedures, prior/concomitant medications). All potential sites of tumor lesions are assessed initially by radiologic techniques, or if appropriate, by skin color photography (e.g., skin lesion). Radiologic techniques at screening include: CT/MRI for the chest, abdomen and pelvis, whole body bone scan (if clinically indicated), bone x-ray, CT or MRI (if skeletal abnormalities identified by bone scan), brain CT/MRI (if clinically indicated), and any other imaging method (if clinically indicated). The same imaging methodology is used to evaluate a specific lesion throughout the study.

Patients may voluntarily withdraw from the study treatment or be removed at the investigator's decision. Patients must be withdrawn from the study treatment for reasons of death or pregnancy. Patients may be withdrawn from the study if any of the following occur: adverse event, lost to follow-up, physician decision, progressive disease, protocol deviation, study terminated, technical problems, subject/guardian decision, adjustment to study treatment that result in discontinuation, use of prohibited medications, or interruption of study treatment for >28 days from the intended day of the next scheduled dose.

In Dose Escalation Phase I, everolimus is administered orally at a starting dose of 2.5 mg once daily on Day 1. COMPOUND A is administered orally at a starting dose of 300 mg once daily starting on Day 8 in a 28-day cycle. A complete treatment cycle is defined as 28 days during which COMPOUND A and everolimus are given once daily.

For dose escalation, each cohort is consisting of 3 to 6 evaluable patients who are treated at the specified dose level. Initially, all patients are treated with the double combination at the starting dose level. The following table (Table 1-1) describes the starting dose and the dose levels that may be evaluated in this study:

| Dose levels | COMPOUND A | Everolimus |
|---|---|---|
| Dose Level −1a* | 250 mg | 2.5 mg |
| Dose Level −1b* | 250 mg | 5 mg |
| Dose Level 1 | 300 mg | 2.5 mg |
| Dose Level 2 | 300 mg | 5 mg |
| Dose Level 3a** | 300 mg | 10 mg |
| Dose Level 3b** | 350 mg | 5 mg |
| Dose Level 4 | 350 mg | 10 mg |

*Dose levels −1a and −1b represent dose de-escalation from the starting dose level. Two different “DL-1” may be explored depending on PK outcomes: DL-1a (COMPOUND A 250 mg and Everolimus 2.5 mg) is explored if everolimus PK overexposure is seen at DL1. DL-1b (COMPOUND A 250 mg and Everolimus 5 mg) is explored in the absence of significant increase of everolimus exposure at DL1. No dose de-escalation below dose level −1 is permitted for this study.
**After dose level 2 (COMPOUND A 300 mg/Everolimus 5 mg) is studied, further escalation is occurring at dose levels 3a and 3b in parallel until MTD and/or RDE has been established for COMPOUND A and Everolimus combination. The intention for exploring the DL-3b is to evaluate the potential DDI effect between the two drugs at the theoretical MTD of COMPOUND A (350 mg).

Assuming that dose level 1 is confirmed to be feasible at the prior derivation stage, doses are escalated in cohorts of newly enrolled 3-6 patients. After dose level 2 has been studied, further escalation is occurring at dose levels 3a and 3b in parallel. The dose is escalated until MTD/RDE is determined.

The MTD is defined as the highest combination drug dosage not causing medically unacceptable dose limiting toxicities (DLT) in more than 35% of the treated patients in the first 35 days of treatment. Dose escalation/de-escalation is guided using Bayesian Logistic Regression Model (BLRM) with overdose control. The recommendation from the Bayesian analysis and other study information (e.g, overall toxicity, PK, efficacy) is evaluated before dose escalation or de-escalation. Typically, the MTD is a tested dose with maximum probability of targeted toxicity (DLT rate between 16%-35%). The use of EWOC principle limits the risk that a potential next dose will exceed the MTD.

A DLT is defined as an adverse event or abnormal laboratory value assessed as unrelated to disease, disease progression, inter-current illness, or concomitant medications that occurs within the first 35 days of treatment and meets any of the criteria included in the following table (Table 1-2):

| TOXICITY | DLT CRITERIA |
|---|---|
| Blood and lymphatic | Febrile neutropenia CTCAE Grade ≥ 3 |
| system disorders | |
| Cardiac disorders | Cardiac toxicity CTCAE Grade ≥ 3 or cardiac event that is symptomatic or |
| | requires medical intervention |
| | Clinical signs of cardiac disease, such as unstable angina or myocardial |
| | infarction, or Troponin CTCAE Grade 3 (confirmed with a repeat Troponin |
| | within 24 hrs) |
| | ECG QTc interval prolonged CTCAE Grade ≥ 3 |
| Vascular disorders | Persistent hypertension CTCAE Grade ≥ 3 requiring more than one drug |
| Hypertension | or more intensive therapy than previously administered. |
| General disorders and | Fatigue CTCAE Grade ≥ 3 for >7 consecutive days |
| administration site | |
| conditions | |
| Skin and subcutaneous | Rash or photosensitivity CTCAE Grade 3 for >7 consecutive days despite |
| tissue disorders ^{a}: | skin |
| Rash and/or | toxicity treatment |
| photosensitivity | Rash or photosensitivity CTCAE Grade 4 |
| Metabolism and nutrition | Hyperglycemia Grade 2 (FPG 200-249 mg/dL; 11.2-13.8 mmol/L) |
| disorders: | (confirmed with a repeat FPG within 24 hrs) that does not resolve to |
| Hyperglycemia^{b} | grade 0 (<140 mg/dL; <7.8 mmol/L) within 14 consecutive days (after |
| | initiation of oral anti-diabetic treatment |
| | Hyperglycemia Grade 3 (FPG 250-399 mg/dL; 13.9-22.2 mmol/L) |
| | (confirmed with a repeat FPG within 24 hrs) for >7 consecutive days |
| | despite oral antidiabetic treatment |
| | Hyperglycemia Grade 4 (FPG ≥ 400 mg/dL; ≥22.3 mmol/L) |
| | Hyperglycemia leading to diabetic keto-acidosis, hospitalization for IV |
| | insulin infusion, or non-ketotic coma |
| Neuropathy/pain | ≥CTCAE grade 3 peripheral sensory or motor neuropathy |
| GI disorders ^{a} | Diarrhea CTCAE Grade ≥ 3 ≥ 48 hrs., despite the use of anti-diarrhea |
| | therapy |
| | Nausea/vomiting CTCAE Grade ≥ 3 ≥ 48 hrs., despite the use of anti- |
| | emetic therapy |
| | Pancreatitis CTCAE Grade ≥ 3 |
| Investigations^{c} | Blood bilirubin^{d} CTCAE Grade 2 for >7 consecutive days |
| | Blood bilirubin^{d} CTCAE Grade ≥ 3 |
| | AST or ALT CTCAE Grade ≥ 3 in conjunction with blood bilirubin^{d} CTCAE |
| | Grade ≥ 2 of any duration |
| | AST or ALT CTCAE Grade ≥ 3 for >7 consecutive days |
| | AST or ALT CTCAE Grade 4 |
| | Serum alkaline phosphatase CTCAE Grade 4 for >7 consecutive days |
| | Serum lipase and/or serum amylase (asymptomatic) CTCAE Grade 3 for |
| | >7 consecutive days |
| | Serum lipase and/or serum amylase (asymptomatic) CTCAE Grade 4 |
| | Serum creatinine CTCAE Grade ≥ 3 |
| | Neutrophil count CTCAE Grade ≥ 3 for >7 consecutive days |
| | Platelet count CTCAE Grade 3 for >7 consecutive days and/or with signs |
| | of bleeding |
| | Platelet count CTCAE Grade 4 |
| | Hypomagnesaemia CTCAE Grade 3 for >3 consecutive days and not |
| | correctable with supplements, or symptomatic |
| | Hypomagnesaemia CTCAE Grade 4 |
| Stomatitis/mucositis | CTCAE Grade 2 for >7 consecutive days |
| | CTCAE Grade ≥ Grade 3 |
| Hepatitis B reactivation | For patients with baseline negative HBV-DNA and HbsAg AND positive |
| | HBs Ab (with no prior history of vaccination against HBV), OR positive |
| | HBc Ab, reactivation is defined as new appearance of measurable HBV- |
| | DNA |
| Hepatitis C reactivation | For patients with baseline knowledge of past hepatitis C infection with no |
| | detectable HCV-RNA, reactivation is defined as new appearance of |
| | detectable HCV-RNA |
| Other hematologic & non- | Any other CTCAE Grade ≥ 3 toxicity except: |
| hematologic toxicities^{e} | Lymphocyte count decreased (lymphopenia) CTCAE Grade ≥ 3 unless |
| | clinically significant |

^{a} Patients do not initially receive prophylactic treatment for skin toxicity or nausea/vomiting during Cycle 1. However, prophylactic treatment may be initiated in all patients at the dose level where these toxicities have been observed and in all further patients if at least 1 patient has experienced skin toxicity or nausea/vomiting CTCAE Grade ≥ 3 or if at least 2 patients experienced skin toxicity or nausea/vomiting CTCAE Grade ≥ 2. However anti-emetics may be applied for treatment if the patient has experienced nausea/vomiting CTCAE Grade ≥ 1, at the discretion of the physician.
^{b}Not according to CTCAEv4.03. Of note: Hyperglycemia occurring during corticosteroids administration is considered DLT if not resolved within 2 days after the end of corticosteroid treatment.
^{c}For any CTCAE Grade 3 or 4 hepatic toxicity that does not resolve within 7 days to CTCAE Grade ≤ 1 (or CTCAE Grade ≤ 2 if liver infiltration with tumor present), an abdominal CT scan must be performed to assess if it is related to disease progression.
^{d}Refers to total bilirubin
^{e}with the exception of alopecia
Apart from the criteria listed above, if a lower grade AE leads to a dose interruption of more than 7 consecutive days of COMPOUND A and/or Everolimus and/or Exemestane when all drugs are supposed to be administered concurrently (Day 8 to Day 35), or more than 7 consecutive days of COMPOUND A and/or everolimus and/or exemestane within the first 35 days (Day 1 to Day 35), this AE is considered as DLT.
Note
that hypersensitivity reactions are not considered as DLT. However patients experiencing severe hypersensitivity reaction during cycle 1 despite adequate premedication should be discontinued from the study.

National Cancer Institute Common Terminology Criteria for Adverse Events (NCI CTCAE) version 4.03 is used for all grading.

Patients must complete a minimum of 35 days of treatment with minimum safety evaluation and drug exposure or have had a DLT within the first 35 days of treatment to be considered evaluable for dose escalation decisions. Dose escalation decisions are made when the cohort of patients has met these criteria. If only 2 of the 3 patients in a cohort are evaluable and neither subject has experienced treatment related toxicity >CTCAE grade 1, dose escalation decisions may be considered.

If the first 2 patients in a cohort experience a DLT, further enrollment to that cohort is stopped and the BLRM updated with this new information. Re-evaluation of the available safety, PK and PD data is performed. By incorporating information gained at the preceding dose levels, additional patients may be enrolled at this dose level or a lower dose level if agreed upon and if the BLRM predicts that the risk that this dose exceeds the MTD remains below 25% (EWOC).

For patients who do not tolerate the specified dosing schedule, dose adjustments are permitted to allow the patient to continue the study treatment. All dose modifications are based on the worst preceding toxicity as graded by the NCI-CTCAE version 4.03. For patients who tolerate well the study treatment, dose increase is not permitted. If dose reduction is required, the following table (Table 1-3) defines the dose reduction steps for the double combination of COMPOUND A and everolimus:

Dose reduction^{a}

| | Starting dose | | Reduction step-1 | | Reduction step-2 | |
|---|---|---|---|---|---|---|
| COMPOUND A | 350 | mg | 300 | mg | 250 | mg |
| | 300 | mg | 250 | mg | 200 | mg^{b} |
| | 250 | mg | 200 | mg^{b} | NA | |
| Everolimus | 2.5 | mg^{c} | NA | | NA | |
| | 5 | mg | 2.5 | mg^{c} | NA | |
| | 10 | mg | 5 | mg | 2.5 | mg^{c} |

^{a}Dose reduction should be based on the worst toxicity demonstrated at the last dose
^{b}Dose reduction below 200 mg is not allowed.
^{c}Dose reduction below 2.5 mg is not allowed.

Further, the following table (Table 1-4) defines dosing modification guidelines for non-hematologic toxicities:

| Adverse drug reaction | Toxicity | Action^{a} |
|---|---|---|
| Cardiac-QTc | QTcF > 500 ms | First Occurrence: |
| prolongation | (≥Grade 3) | Interrupt COMPOUND A and/or everolimus |
| | or >60 ms change | Perform a repeat ECG within one hour of the |
| | from baseline | first QTcF of >500 ms or >60 ms from baseline: |
| | on at least two | if QTcF remains >500 ms or >60 ms from |
| | separate ECGs | baseline, repeat ECG as clinically indicated, but |
| | | at least once a day until the QTcF returns to |
| | | <480 ms. |
| | | Seek cardiologist input; address electrolytes, |
| | | calcium and magnesium abnormalities; |
| | | concomitant medication must be reviewed. |
| | | Once QTcF prolongation has resolved, |
| | | COMPOUND A and/or everolimus may be |
| | | restarted at a one lower dose level |
| | | Second Occurrence: |
| | | Permanently discontinue COMPOUND A |
| | | and/or everolimus |
| Cardiac-Left Ventricular | Asymptomatic, resting | Maintain dose level, and continue |
| Systolic dysfunction | ejection fraction 40- | COMPOUND A and/or everolimus with caution |
| | 50%; or 10-20% drop | Repeat LVEF within 4 weeks or as clinically |
| | from baseline | appropriate |
| | Symptomatic, | Interrupt COMPOUND A and/or everolimus |
| | responsive to | until resolved (patient is asymptomatic, has a |
| | intervention, ejection | resting ejection fraction ≥40% and ≥20% |
| | fraction 20-39% or | decrease from baseline), then decrease by 1 |
| | >20% drop from | dose level |
| | baseline | |
| | Refractory or poorly | Permanently discontinue COMPOUND A |
| | controlled, ejection | and/or everolimus |
| | fraction < 20% | |
| Diarrhea | Grade 1 | Maintain dose level |
| | Grade 2 | Interrupt COMPOUND A and/or everolimus |
| | | dose until resolved to ≤Grade 1, then restart |
| | | COMPOUND A and/or everolimus at same |
| | | dose |
| | ≥Grade 3 | Interrupt COMPOUND A and/or everolimus |
| | | dose until resolved to ≤Grade 1, then reduce |
| | | COMPOUND A and/or everolimus 1 dose level |
| Eyes disorders | ≥Grade 3 | Permanently discontinue COMPOUND A |
| | ocular/vision | and/or everolimus |
| | symptoms interfering | |
| | with activities of daily | |
| | life or requiring | |
| | medical intervention | |
| Hepatic¹-Bilirubin | Grade 1 (>ULN-1.5 × | Maintain dose level with LFTs² monitored as |
| (²for patients with Gilbert | ULN) | per protocol |
| Syndrome these dose | Grade 2 (>1.5-3.0 × | Interrupt COMPOUND A and/or everolimus |
| modifications apply to | ULN) with ALT or | until resolved to ≤Grade 1, then: |
| changes in direct bilirubin | AST ≤ 3.0 × ULN | If treatment delay is ≤7 days, restart |
| only) | | COMPOUND A and/or everolimus at same |
| | | dose |
| | | If resolved in >7 days, decrease COMPOUND A |
| | | and/or everolimus by one dose level |
| | Grade 3 (>3.0-10.0 × | Interrupt COMPOUND A and/or everolimus |
| | ULN) with ALT or | dose until resolved to ≤Grade 1, then: |
| | AST ≤ 3.0 × ULN | If treatment delay is ≤7 days, restart |
| | | COMPOUND A and/or everolimus at same |
| | | dose |
| | | If resolved in >7 days, permanently |
| | | discontinue patient from COMPOUND A |
| | | and/or everolimus |
| | Grade 4 (>10.0 × ULN) | Permanently discontinue patient from |
| | | COMPOUND A and/or everolimus |
| Hepatic¹-AST or ALT | Grade 1 (>ULN-3.0 × | Maintain current COMPOUND A and/or |
| | ULN) | everolimus dose level with LFTs monitored per |
| | | protocol |
| | Grade 2 (>3.0-5.0 × | For patients with grade 0 or 1 at screening: |
| | ULN) without total | Omit COMPOUND A and/or everolimus dose |
| | bilirubin elevation to | until resolved to ≤baseline value |
| | >2.0 × ULN | If treatment delay is ≤7 days, restart at same |
| | | dose |
| | | If resolved in >7 days, decrease COMPOUND A |
| | | and/or everolimus by 1 dose level |
| | | For patients with grade 2 at screening: |
| | | maintain dose level with LFTs monitored as |
| | | per protocol |
| | Grade 3 (>5.0-20.0 | Interrupt COMPOUND A and/or everolimus |
| | ULN) without total | administration until resolution to Baseline |
| | bilirubin elevation to | value |
| | >2.0 × ULN | If resolution occurs ≤7 days, COMPOUND A |
| | | and/or everolimus should be re-started at the |
| | | same dose level. |
| | | If resolution takes >7 days, or if event recurs |
| | | within 28 days, hold COMPOUND A and/or |
| | | everolimus until recovery to ≤grade 1 or |
| | | baseline grade/value and reintroduce |
| | | COMPOUND A and/or everolimus at one dose |
| | | level lower, if available. |
| | Grade 4 (>20 × ULN) | Interrupt COMPOUND A and/or everolimus |
| | without bilirubin | administration until resolution to ≤grade 1. If |
| | elevation to >2.0 × | resolution occurs ≤7 days, COMPOUND A |
| | ULN | and/or everolimus should be re-started at one |
| | | dose level lower. If resolution takes >7 days, |
| | | discontinue study drug. |
| | Recurrence of grade 4 | Discontinue COMPOUND A and/or everolimus. |
| | after dose reduction | |
| | or toxicity requiring | |
| | study drug | |
| | interruption for >28 | |
| | days | |
| Hepatic¹-AST | AST or ALT > 3.0 × ULN | Permanently discontinue COMPOUND A |
| or ALT and | and total bilirubin > | and/or everolimus |
| concurrent | 2.0 × ULN | |
| Bilirubin | | |
| Clinical liver failure | Grade 3 or 4 | Permanently discontinue COMPOUND A |
| (asterixis or | | and/or everolimus |
| encephalopathy/coma) | | |
| Reactivation of HBV or | | See below |
| HCV flare | | |
| Hyperglycemia | Grade 1 (>ULN-160 | Maintain COMPOUND A and/or everolimus |
| | mg/dL) [>ULN-8.9 | dosing at the current dose level |
| | mmol/L] confirmed | As per investigator's discretion, initiate or |
| | within 24 hours | intensify medication with appropriate anti- |
| | | diabetic treatment such as oral anti- |
| | | hyperglycemic therapy (e.g. metformin) |
| | | Check FPG as clinically indicated and at least |
| | | weekly for 8 weeks, then continue checking at |
| | | least every 2 weeks |
| | Asymptomatic grade 2 | Maintain COMPOUND A and/or everolimus |
| | (>160-250 mg/dL) | dose level and re-check within 24 hours: if |
| | [>8.9-13.9 mmol/L] | grade worsens or improves, follow specific |
| | | recommendations; if grading is confirmed: |
| | | Continue COMPOUND A and/or everolimus |
| | | dosing. |
| | | Initiate or intensify medication with |
| | | appropriate anti-diabetic treatment such as |
| | | oral anti-hyperglycemic therapy (e.g. |
| | | metformin) as per investigator's discretion; |
| | | consider adding a second oral agent if no |
| | | improvement after several days |
| | | Monitor FPG as clinically indicated and at least |
| | | weekly until FPG resolves to ≤Grade 1 |
| | | If FPG does not resolve to ≤Grade 1 within 14 |
| | | days after institution of appropriate anti- |
| | | diabetic treatment, reduce COMPOUND A |
| | | and/or everolimus by 1 dose level |
| | | Continue with anti-diabetic treatment and |
| | | check FPG at least weekly for 8 weeks, then |
| | | continue checking at least every 2 weeks |
| | Asymptomatic grade 3 | Interrupt COMPOUND A and/or everolimus |
| | (>250-500 mg/dL) | and re-check within 24 hours: |
| | [>13.9- 27.8 mmol/L] | If grade worsens or improves, follow specific |
| | | recommendations. |
| | Or | If grading is confirmed, interrupt COMPOUND |
| | | A and/or everolimus dose. |
| | Grade 2 with signs or | Consider administering intravenous hydration |
| | symptoms of | and intervention for |
| | hyperglycemia (e.g., | electrolyte/ketoacidosis/hyperosmolar |
| | mental status | disturbances as clinically appropriate. |
| | changes, excessive | Initiate or intensify medication with |
| | thirst, polyuria) | appropriate antidiabetic treatment (consider |
| | | adding insulin) as per investigator's discretion. |
| | | Monitor FPG as clinically indicated and at least |
| | | twice weekly until FPG resolves to ≤Grade 1 |
| | | If FPG resolves to Grade1 within 14 days, then |
| | | re-start COMPOUND A and/or everolimus with |
| | | dose reduction by 1 dose level |
| | | If FPG doesn't resolve to Grade1 within 14 |
| | | days, then permanently discontinue |
| | | COMPOUND A and/or everolimus |
| | | Continue with anti-diabetic treatment and |
| | | check FPG at least weekly for 8 weeks, then |
| | | continue checking at least every 2 weeks |
| | Grade 4 (>500 mg/dL) | Interrupt COMPOUND A and/or everolimus, |
| | [≥27.8 mmol/L] | initiate or intensify medication with |
| | Or Grade 3 with signs | appropriate anti-diabetic treatment (consider |
| | or symptoms of | adding insulin), re-check within 24 hours. |
| | hyperglycemia (for | If grade improves then follow specific grade |
| | ex., mental status | recommendations. |
| | changes, excessive | If FPG is confirmed at Grade 4, permanently |
| | thirst, polyuria) | discontinue COMPOUND A and/or everolimus |
| | | Administer intravenous hydration and |
| | | intervention for |
| | | electrolyte/ketoacidosis/hyperosmolar |
| | | disturbances as clinically appropriate |
| | | Initiate or intensify medication with |
| | | appropriate anti-diabetic treatment (consider |
| | | adding insulin) as per investigator's discretion |
| | | Check FPG at least weekly for 8 weeks, then |
| | | continue checking at least every 2 weeks if |
| | | clinically indicated |
| Mucositis | Grade 1 or Tolerable | Maintain dose level. |
| | Grade 2 | Non-alcoholic or salt water mouth wash. |
| | Intolerable Grade 2 or | First occurrence: interrupt COMPOUND A |
| | Grade 3 | and/or everolimus until ≤Grade 1 and |
| | | decrease COMPOUND A and/or everolimus by |
| | | 1 dose level |
| | | (if stomatitis is readily manageable with |
| | | optimal management, re-introduction at the |
| | | same level might be considered at the |
| | | discretion of the investigator). |
| | | Second occurrence: interrupt COMPOUND A |
| | | and/or everolimus until ≤Grade 1 and |
| | | COMPOUND A and/or everolimus 1 dose level. |
| | Grade 4 | Permanently discontinue patient from |
| | | COMPOUND A and/or everolimus |
| Pancreatitis | Grade ≥ 3 | Permanently discontinue COMPOUND A |
| | | and/or everolimus |
| Photosensitivity | Grade 1 | Maintain dose level |
| | Grade 2 | Interrupt COMPOUND A and/or everolimus |
| | | dose until resolved to ≤Grade 1 then: |
| | | If resolved in ≤7 days, reduce COMPOUND A |
| | | and/or everolimus by one dose level |
| | | If resolved in >7 days, permanently |
| | | discontinue COMPOUND A and/or everolimus |
| | Grade ≥ 3 | Permanently discontinue COMPOUND A |
| | | and/or everolimus |
| Pneumonitis | | Please see below. |
| Pruritus³ | Grade 1 | Maintain dose level. Consider to start |
| | | alternate treatment with topical steroid |
| | (Mild or localized | moderate strength or topical antipruritics |
| | pruritus) | applied twice daily |
| | Grade 2 | Tolerable: same management as Grade 1 |
| | (Intense or | Intolerable: |
| | widespread; | First occurrence: Omit COMPOUND A |
| | intermittent; skin | and/or everolimus dose until resolved to |
| | changes from | Grade ≤ 1 then: |
| | scratching; limiting | If resolved in ≤2 weeks, maintain dose |
| | instrumental ADL) | level. |
| | | If resolved in more than 2 weeks, reduce |
| | | by 1 dose level. |
| | | Start recommended treatment with topical |
| | | steroids moderate potency or topical |
| | | antipruritics twice daily and add oral |
| | | antihistamines |
| | | Second occurrence: reduce by 1 dose level |
| | | Initiate or intensify recommended treatment |
| | | as described above |
| | Grade 3 | Interrupt COMPOUND A and/or everolimus |
| | (Intense or | dose until resolved to CTCAE Grade ≤ 1; then |
| | widespread; constant; | reduce by 1 dose level. |
| | limiting self-care ADL | Start recommended treatment with: oral |
| | or sleep) | corticosteroids or oral antihistamines in |
| | | association with GABA agonists³ |
| | Grade 4 | Discontinue COMPOUND A and/or everolimus |
| Rash maculo-papular | Grade 1 | Maintain COMPOUND A and/or everolimus |
| | (macules or papules | dose level. Consider to initiate alternate |
| | covering <10% BSA | treatment with topical steroid bid and oral |
| | with or without | antihistamines. |
| | symptoms) | |
| | Grade 2 (macules or | Tolerable: same management as grade 1 |
| | papules covering 10- | Intolerable: Start recommended treatment³ |
| | 30% BSA with or | with topical steroids bid, oral antihistamines |
| | without symptoms) | and oral steroids. |
| | | Treatment can be continued up to 2 weeks |
| | | after rechallenge with COMPOUND A; consider |
| | | prompt implementation in case of flare after |
| | | interruption of recommended treatment. |
| | | First occurrence: Omit dose until resolved |
| | | to Grade ≤ 1 then: |
| | | If resolved in ≤2 weeks, maintain dose |
| | | level. |
| | | If resolved in more than 2 weeks, reduce |
| | | by 1 dose level. |
| | | Second occurrence: reduce by 1 dose level |
| | Grade 3 (macules or | Interrupt dose until resolved to CTCAE |
| | papules covering | Grade ≤ 1; then reduce by 1 dose level. |
| | >30% BSA with or | |
| | without symptoms) | |
| | | Start recommended treatment³ with topical |
| | | steroids bid, oral antihistamines and oral |
| | | steroids. Treatment can be continued up to 2 |
| | | weeks after rechallenge with everolimus |
| | | and/or COMPOUND A; consider prompt |
| | | implementation in case of flare after |
| | | interruption of recommended treatment |
| | Grade 4 (Papules | Permanently discontinue COMPOUND A |
| | and/or pustules | and/or everolimus |
| | covering any % BSA, | According to the investigator's discretion, a |
| | associated with | paired skin biopsy could be obtained (from |
| | symptoms or not but | both an affected and an unaffected skin area |
| | associated with | for local histopathology assessment) if |
| | extensive | clinically appropriate. |
| | superinfection) | |
| Rash (acneiform) | Grade 1 | Maintain COMPOUND A and/or everolimus |
| | (<10% body surface | dose level. Consider to initiate alternate |
| | area BSA; no | treatment with topical steroid moderate |
| | associated erythema | potency and topical antibiotic bid |
| | or pruritus) | |
| | Grade 2 | Tolerable: same management as grade 1 |
| | (10 to 30% BSA and | Intolerable: |
| | associated with | First occurrence: Omit COMPOUND A and/or |
| | erythema or pruritus; | everolimus dose until resolved to Grade ≤ 1 |
| | limited instrumental | then: |
| | activities of daily living | If resolved in ≤2 weeks, maintain COMPOUND |
| | (ADL) | A and/or everolimus dose level. |
| | | If resolved in more than 2 weeks, reduce by 1 |
| | | COMPOUND A and/or everolimus dose level. |
| | | Start recommended treatment** oral |
| | | antibiotic for 6 weeks; stop topical antibiotics |
| | | if being used and start with topical steroids of |
| | | moderate potency |
| | | Second occurrence: reduce by COMPOUND A |
| | | and/or everolimus by 1 dose level |
| | | Initiate or intensify recommended treatment |
| | | as described above |
| | Grade 3 | Omit COMPOUND A and/or everolimus dose |
| | (>30% BSA And | until resolved to CTCAE Grade ≤ 1; then reduce |
| | associated with | COMPOUND A and/or everolimus by 1 dose |
| | pruritus; | level. |
| | limiting self ADL) | |
| | | Start alternate treatment with oral antibiotic |
| | | for 6 weeks. If infection suspected (yellow |
| | | crusts, purulent discharge, painful skin/nares) |
| | | then switch to broad spectrum/gram negative |
| | | antibiotics; consider skin swab for bacterial |
| | | culture. Add topical steroid of moderate |
| | | potency. |
| | Grade 4 | Permanently discontinue patient from |
| | Papules and/or | COMPOUND A and/or everolimus |
| | pustules covering | |
| | any % BSA, associated | |
| | with symptoms or not | |
| | but associated with | |
| | extensive | |
| | superinfection | |
| Serum creatinine | <2 × ULN | Maintain COMPOUND A and/or everolimus |
| | | dose level |
| | 2-3 × ULN | Interrupt COMPOUND A and/or everolimus |
| | | dose until resolved to grade ≤ 1, then: |
| | | If treatment delay is ≤7 days, restart |
| | | COMPOUND A and/or everolimus at same |
| | | dose |
| | | If resolved in >7 days, then reduce |
| | | COMPOUND A and/or everolimus by 1 dose |
| | | level |
| | Grade 3 (>3.0-6.0 × | Permanently discontinue COMPOUND A |
| | ULN) | and/or everolimus |
| | Grade 4 (>6.0 × ULN) | Permanently discontinue COMPOUND A |
| | | and/or everolimus |
| All other adverse events | Grade 1 or 2 | Maintain dose level |
| | Grade 3 | Interrupt COMPOUND A and/or everolimus |
| | | dose until resolved to ≤Grade 1, then |
| | | decrease COMPOUND A and/or everolimus by |
| | | one dose level |
| | | Note: Interrupt dose for ≥Grade 3 vomiting or |
| | | Grade 3 nausea only if the vomiting or nausea |
| | | cannot be controlled with optimal antiemetic |
| | Grade 4 | Permanently discontinue patient from |
| | | COMPOUND A and/or everolimus |
| Any non-hematologic | Any grade and | Permanently discontinue COMPOUND A |
| toxicity | requiring study drug | and/or everolimus |
| | interruption for >28 | |
| | days | |

¹Should HCV flare be confirmed, the guidelines for flare must take precedence
²LFTs include albumin, ALT, AST, total bilirubin (fractionated if total bilirubin > 2.0 × ULN), alkaline phosphatase (fractionated if alkaline phosphatase is grade 2 or higher) and GGT Patients with grade 0 or 1 at screening experiencing ALT/AST/bilirubin increase ≥ grade 2 the liver function tests must be monitored weekly or more frequently if clinically indicated until resolved to ≤grade 1
In case of any occurrence of ALT/AST/bilirubin² increase ≥ grade 3 the liver function tests must be monitored weekly or more frequently if clinically indicated until resolved to ≤grade 1; hereafter the monitoring should be continued every other week or more frequently if clinically indicated until the end of treatment with study medication
Patients who discontinued study treatment should be monitored weekly, including LFTs² or more frequently if clinically indicated until resolved to ≤grade 1 or stabilization (no CTCAE grade change over 4 weeks).
For the combination of COMPOUND A and/or everolimus, hyperglycemia usually resolves within a few days after COMPOUND A and/or everolimus interruption; temporary interruption of COMPOUND A and/or everolimus may be considered as clinically indicated to improve control of hyperglycemia. Special attention should be paid to the risk of hypoglycemia in patients interrupting COMPOUND A and/or everolimus treatment and receiving insulin or sulfonylurea.
For all grades: instruct patient to follow dietary guidelines according to local and/or institutional standards for management of diabetes mellitus (such as those provided by the American Diabetes Association) during the study
^{a}Daily dose of COMPOUND A cannot be decreased below 200 mg/day. Daily dose of everolimus cannot be decreased by 2.5 mg/day.

Further, the following table (Table 1-5) provides the dosing guidelines for hematologic toxicities:

| Adverse drug reaction | Severity | Action^{a} |
|---|---|---|
| Thrombocytopenia | Grade 2 | No action |
| | Grade 3 | Interrupt COMPOUND A and/or |
| | | everolimus until resolution to grade ≤1 |
| | | If resolution occurs ≤7 days, reintroduce |
| | | COMPOUND A and/or everolimus at the |
| | | dose level prior to interruption. |
| | | If resolution occurs >7 days, or event |
| | | occurs within 28 days, reintroduce |
| | | COMPOUND A and/or everolimus at one |
| | | dose level lower, if available. |
| | Grade 4 | Interrupt COMPOUND A and/or |
| | | everolimus until recovery to grade ≤1. |
| | | Then reintroduce everolimus at one dose |
| | | level lower, if available. |
| Neutropenia or Anemia | Grade 3 | Interrupt COMPOUND A and/or |
| | | everolimus until resolution to grade ≤1 or |
| | | baseline value |
| | | If AE resolution occurs ≤7 days, |
| | | reintroduce COMPOUND A and/or |
| | | everolimus at the same dose level. |
| | | If AE resolution occurs >7 days, or event |
| | | occurs within 28 days, reintroduce |
| | | COMPOUND A and/or everolimus at one |
| | | dose level lower, if available. |
| | Grade 4 | Interrupt COMPOUND A and/or |
| | | everolimus until recovery to grade ≤1 or |
| | | baseline value. Reintroduce COMPOUND A |
| | | and/or everolimus at one dose level lower, |
| | | if available. |
| Febrile neutropenia | | Interrupt COMPOUND A and/or |
| | | everolimus until resolution to grade ≤1 |
| | | (or baseline value) and no fever. |
| | | Reintroduce COMPOUND A and/or |
| | | everolimus at one dose level lower, if |
| | | available. |
| All other adverse event | Recurrence of grade 3 | Reduce COMPOUND A and/or everolimus |
| | toxicity after dose | dose to the next lower dose level |
| | reduction | |
| | Recurrence of grade 4 | Discontinue COMPOUND A and/or |
| | toxicity (including febrile | everolimus |
| | neutropenia) after dose | |
| | reduction | |
| Any hematologic toxicity | Any grade and requiring | Discontinue COMPOUND A and/or |
| | study drug interruption | everolimus |
| | for >28 days | |

^{a}Daily dose of COMPOUND A cannot be decreased below 200 mg/day. Daily dose of everolimus cannot be decreased by 2.5 mg/day.

If diagnosed with an invasive systemic fungal infection, patients should be discontinued from everolimus and treated with appropriate antifungal therapy.

If diagnosed with a non-infectious pneumonitis, patients should be treated as defined in the following table (Table 1-6) for management:

| Worst grade | | | | COMPOUND A dose |
|---|---|---|---|---|
| pneumonitis | Required investigations | Management of pneumonitis | Everolimus dose adjustment | adjustment |
| Grade 1 | CT scans with lung windows. | No specific therapy is | No dose adjustment required. | No dose adjustment |
| | Repeat at least every | required | Initiate appropriate monitoring. | required (Administer |
| | 8 weeks until return to | | | 100% of COMPOUND A). |
| | within normal limits. | | | |
| Grade 2 | CT scan with lung windows. | Symptomatic only. | Rule out infection and consider | Reduce COMPOUND A |
| | Consider pulmonary function | Consider corticosteroids | interruption of everolimus until | dose by 1 dose level ^{b} until |
| | testing includes: spirometry, D_{L}CO, | and/or other supportive | symptoms improve to Grade ≤1. | recovery to ≤Grade 1. |
| | and room air O₂ | therapy if symptoms are | Re-initiate everolimus at one | COMPOUND A may also be |
| | saturation at rest. | troublesome. | dose level lower ^{a}. | interrupted if symptoms |
| | Consider a bronchoscopy | | Discontinue everolimus if | are troublesome. |
| | with biopsy and/or BAL. | | failure to recover | Patients will discontinue |
| | Monitoring at each cycle until | | within ≤28 days. | COMPOUND A if they fail to |
| | return to ≤grade | | | recover to ≤Grade 1 within |
| | 1. Return to initial monitoring | | | 28 days. |
| | frequency if no recurrence. | | | |
| Grade 3 | CT scan with lung windows and | Consider | Rule out infection and | Hold treatment with |
| | pulmonary function testing | corticosteroids if | interrupt everolimus until | COMPOUND A until recovery |
| | includes: spirometry, DLCO, | infective origin is | symptoms improve to Grade ≤1. | to ≤Grade 1. |
| | and room air 02 saturation | ruled out. Taper | Consider reinitiating everolimus | May restart COMPOUND A |
| | at rest. | as medically | at one dose level lower ^{a}. | within 28 days at a reduced dose |
| | Monitoring at each cycle until | indicated. | Discontinue everolimus if | (by one level) ^{b} if |
| | return to ≤grade | | failure to recover | evidence of clinical benefit |
| | 1. Return to initial monitoring | | within ≤28 days. | |
| | frequency if no recurrence. | | If toxicity recurs at grade 3, | |
| | Bronchoscopy with biopsy | | consider discontinuation of | |
| | and/or BAL is recommended. | | everolimus | |
| Grade 4 | CT scan with lung windows and | Consider | Rule out infection and | Discontinue treatment with |
| | required pulmonary function | corticosteroids if | discontinue everolimus. | COMPOUND A. |
| | testing, if possible, includes: | infective origin is | | |
| | spirometry, DLCO, and room air 02 | ruled out. Taper | | |
| | saturation at rest. | as medically | | |
| | Monitoring at each cycle until | indicated. | | |
| | return to ≤grade | | | |
| | 1. Return to initial monitoring | | | |
| | frequency if no recurrence. | | | |
| | Bronchoscopy with biopsy | | | |
| | and/or BAL is recommended if | | | |
| | possible. | | | |

^{a} Daily dose of everolimus cannot be decreased below 2.5 mg/day
^{b} Daily dose of COMPOUND A cannot be decreased below 200 mg/day

A diagnosis of non-infections pneumonitis is considered in patients presenting with non-specific respiratory signs and symptoms such as hypoxia, pleural effusion, cough or dyspnea, and in whom infectious, neoplastic and other non-medicinal causes have been excluded. Patients who develop radiological changes suggestive of non-infectious pneumonitis and have few or no symptoms may continue everolimus therapy without dose alteration.

If diagnosed with stomatitis, oral mucositis, or mouth ulcers, patients should be treated in accordance with the following guidelines:
1. For mild toxicity (grade 1), use conservative measures such as non-alcoholic mouth wash or salt water (0.9%) mouth wash several times a day until resolution.
2. For more severe toxicity (grade 2 in which case patients have pain but are able to maintain adequate oral alimentation, or grade 3 in which case patients cannot maintain adequate oral alimentation), the suggested treatments are topical analgesic mouth treatments (i.e., local anesthetics such as, benzocaine, butyl aminobenzoate, tetracaine hydrochloride, menthol, or phenol) with or without topical corticosteroids, such as triamcinolone oral paste 0.1% (Kenalog in Orabase®).
3. Agents containing alcohol, hydrogen peroxide, iodine, and thyme derivatives may tend to worsen mouth ulcers. It is preferable to avoid these agents.
4. Antifungal agents should be avoided unless a fungal infection is diagnosed. In particular, systemic imidazole antifungal agents (ketoconazole, fluconazole, itraconazole, etc.) should be avoided in all patients due to their strong inhibition of Everolimus metabolism, therefore leading to higher Everolimus exposures. Therefore, topical antifungal agents are preferred if an infection is diagnosed.

For monitoring and prophylactic treatment of hepatitis B reactivation, the following table (Table 1-7) outlines the actions to be taken based on screening hepatitis B results:

| Test | Result | Result | Result | Result | Result |
|---|---|---|---|---|---|
| HBV-DNA | + | +or− | − | − | − |
| HBsAg | +or− | + | − | − | − |
| HBsAb | +or− | +or− | +and | +or− | −or+ |
| | | | no prior | | with prior |
| | | | HBV vaccination | | HBV vaccination |
| HBcAb | +or− | +or− | +or− | + | − |

| | | | |
|---|---|---|---|
| Recommendation | Prophylaxis treatment | No prophylaxis | No specific |
| | should be started 1-2 | Monitor HBV-DNA | action |
| | weeks prior to first dose of | approximately every 4 weeks | |
| | study drug | | |
| | Monitor HBV-DNA | | |
| | approximately every 4 | | |
| | weeks | | |

For HBV V reactivation definition and management guidelines, see the following table (Table 1-8):

HBV reactivation (with or without clinical signs and symptoms)*

| | |
|---|---|
| For patients with baseline | Treat: Start a second antiviral |
| results: | AND |
| Positive HBV-DNA | Interrupt everolimus administration until resolution: |
| OR | ≤baseline HBV-DNA levels |
| positive HBsAg | If resolution occurs within ≤28 days, everolimus should be re-started |
| reactivation is defined as: | at one dose lower, if available. If the patient is already receiving the |
| [Increase of 1 log in HBV- | lowest dose of study drug according to the protocol, the patient |
| DNA relative to baseline | should restart at the same dose after resolution. Both antiviral |
| HBV-DNA value OR new | therapies should continue at least 4 weeks after last dose of study |
| appearance of measurable | drug. |
| HBV-DNA] | If resolution occurs >28 days Patients should discontinue everolimus |
| | but continue both antiviral therapies at least 4 weeks after last dose of |
| | everolimus. |
| For patients with baseline | Treat: Start first antiviral medication |
| results: | AND |
| Negative HBV-DNA and | Interrupt everolimus administration until resolution: |
| HBsAg | ≤undetectable (negative) HBV-DNA levels |
| AND | If resolution occurs within ≤28 days, everolimus should be re-started |
| [Positive HBsAb (with no | at one dose lower, if available. If the patient is already receiving the |
| prior history of vaccination | lowest dose of study drug according to the protocol, the patient |
| against HBV), OR positive | should restart at the same dose after resolution. Antiviral therapy |
| HBcAb] | should continue at least 4 weeks after last dose of everolimus. |
| Reactivation is defined as: | If resolution occurs >28 days Patients should discontinue everolimus |
| New appearance of | but continue antiviral therapy at least 4 weeks after last dose of |
| measurable HBV-DNA | everolimus. |

*All reactivations of HBV are to be defined as grade 3 (e.g. CTCAE Version 4.03-Investigations/Other: Viral Reactivation), unless considered life threatening by the investigator, in which case they should be defined as grade 4. Date of viral reactivation is the date on which the rise or reappearance of HBV-DNA was recorded.

For Hepatitis C (HCV), patients with detectable HCV RNA-PCR testing at screening and patients known to have a history of HCV infection should be monitored every 4 weeks for HCV flare. For definitions of HCV flare ad actions to be taken in event of flare, the following table (Table 1-9) is provided:

| Baseline results | HCV flare definition* | HCV flare management |
|---|---|---|
| Detectable HCV-RNA | >2 log₁₀ IU/mL increase in HCV-RNA | Discontinue everolimus |
| | AND | |
| | ALT elevation > 5 × ULN or 3 × | |
| | baseline level, whichever is higher. | |
| Knowledge of past hepatitis C | New appearance of detectable | Discontinue everolimus |
| infection with no detectable | HCV-RNA | |
| HCV-RNA | AND | |
| | ALT elevation > 5 × ULN or 3 × | |
| | baseline level, whichever is higher. | |

*All flares of HCV are to be recorded as grade 3 (e.g. CTCAE Version 4.03-Investigations-Other: Viral Flare), unless considered life threatening by the investigator; in which case they should be recorded as grade 4. Date of viral flare is the date on which both the clinical criteria described above were met.

If a diagnosis of hyperlipidemia is made, patients should be treated after taking into account the pretreatment status and dietary habits of the patient. Grade 2 or higher hypercholesterolemia (>300 mg/dL or 7.75 mmol/L) or grade 2 hypertriglyceridemia or higher (>2.5× upper normal limit) should be treated with a 3-hydroxy-3-methyl-glutaryl (HMG)-CoA reductase inhibitor (e.g. atorvastatin, pravastatin, fluvastatin) or appropriate triglyceride-lowering medication, in addition to diet.

Patients whose treatment is interrupted or permanently discontinued due to an adverse event or clinically significant laboratory value are followed-up, or if clinically indicated, until resolution of the event, whichever comes first. All patents must be followed up for adverse events or serious adverse events for 30 days following the last dose of study treatment.

If treatment with COMPOUND A and everolimus are interrupted for reasons other than toxicity, treatment with the respective study drug may be resumed at the same dose. The same applies if the patient experienced an unacceptable toxicity set forth above (see dosing guidelines for Dose Escalation Phase I), provided that this toxicity resolved to ≤CTCAE grade 1, unless otherwise specified. If one of the drugs is held for more than 28 days, then this drug should be permanently discontinued and the other drugs may be continued at the same dose as part of the trial therapy at the investigator's discretion, provided the reason is not disease progression.

Dose escalation is continued until identification of the MTD for the expansion phase. This occurs when the following conditions are met:
1. at least 6 evaluable patients have been treated at this dose
2. this dose satisfies one of the following conditions:
   a. the posterior probability of targeted toxicity at this dose exceeds 50% and is the highest among potential doses, or
   b. minimum of 18 evaluable patients have already been treated with the doublet and 6 evaluable patients have already been treated with the triplet during the dose escalation phase.
3. it is the dose recommended for patients, either per the model or by review of all clinical data.

In Dose Escalation Phase II, patients are orally administered everolimus and COMPOUND A at one dose level below MTD of the dual combination with everolimus from the Dose Escalation Phase I. Exemestane is administered at a dose of 25 mg once daily. Everolimus and exemestane are administered orally once daily starting on Day 1, and COMPOUND A is administered once daily starting on Day 8. A complete treatment cycle is defined as 28 days during which COMPOUND A and everolimus are given once daily.

The dose escalation is conducted as described above for Dose Escalation Phase I except as specified hereto. The following table describes the starting dose and the dose levels that may be evaluated in this study:

| Dose levels | COMPOUND A | Everolimus | Exemestane |
|---|---|---|---|
| Dose Level −1* | MTD-1 | MTD-1 | 25 mg |
| Dose Level 1 | MTD-1 | MTD | 25 mg |
| Dose Level 2 | MTD | MTD | 25 mg |

* Dose level −1 represents dose de-escalation from the starting dose level. No dose de-escalation below dose level −1 is permitted for this study.

The starting dose for the first cohort is one dose level lower of the MTD as determined during Dose Escalation Phase I. The COMPOUND A dose may be escalated or de-escalated to the next dose level, as needed, to obtain the MTD/RDE of the triplet. Dose escalation is to the MTD level of COMPOUND A, MTD of everolimus and exemestane 25 mg once daily.

If dose reduction is required, the dose reduction steps described above for Dose Escalation Phase I are followed for COMPOUND A and everolimus. For exemestane, doses may only be modified as disclosed on the package insert of the locally supplied exemestane.

After determination of the MTD/RDE for the triple combination in Dose Expansion Phase II, Expansion Arm A and Expansion Arm B are conducted. Patients are orally administered everolimus, exemestane and COMPOUND A orally once daily starting on Day 1 in a 28-day cycle. COMPOUND A and everolimus are administered at the MTD/RDE determined in Dose Escalation Part II, and exemestane is administered orally once daily at a dose of 25 mg. A complete treatment cycle is defined as 28 days during which COMPOUND A and everolimus are given once daily.

Patients are treated with COMPOUND A, everolimus and exemestane until disease progression (assessed by RECIST 1.1), unacceptable toxicity, death or discontinuation from the study treatment for any other reason (e.g, withdrawal of consent, start of new anti-neoplastic therapy, or at the discretion of the investigator).

During treatment, patients are regularly evaluated for physical examination (including ECOG performance status, height, weight, physical examination, and vital signs), laboratory assessments (including hematology, chemistry (full panel), fasting lipid panel, coagulation, fasting plasma glucose, fasting C-peptide, insulin, HBA1c, lipase, urinalysis, HBV/HCV screening, pregnancy), imaging (including tumor evaluation, 12-lead ECG, ophthalmic evaluation, cardiac imaging, pulmonary function tests), and safety (including adverse events, surgical/medical procedures, prior/concomitant medications, patient diary). Tumor assessments are performed at baseline and then every 8 weeks after study treatment start until disease progression is documented.

In this study, the efficacy endpoints are progression-free survival (PFS), overall response rate (ORR), clinical benefit, and duration of response (DoR). Progression-free survival (PFS) is defined as the time from start date of study treatment to the date of the first documented disease progression or death due to any cause. The distribution of PFS is estimated using Kaplan-Meier methods and results presented with appropriate summary statistics. The proportion of patients PFS events free at 4 months is computed along with exact binomial 90% confidence intervals. The analysis is performed by cohort.

Overall response rate (ORR) is defined as the proportion of patients with a best overall response of complete response (CR) or partial response (PR). The overall response rate and corresponding exact binomial 90% confidence intervals is performed by cohort.

Clinical benefit rate is defined as the proportion of patients with a best overall response of complete response (CR) or partial response (PR) or stable disease (SD) for more than 24 weeks of duration of response. The clinical benefit rate and corresponding exact binomial 90% confidence intervals is performed by cohort.

Duration of response (DoR) is defined as the elapsed time between the date of first documented response (CR or PR) and the following date of event defined as the first documented progression or death due to underlying cancer.

Tumor assessments are done from the screening phase until a progressive disease is documented. If a patient permanently discontinues the study for reasons other than progressive disease or consent withdrawal, tumor evaluations are continued during the post-treatment follow-up phase until a progressive disease is documented or until administration of a new antineoplastic therapy. During Expansion Arm A and Expansion Arm B, tumor evaluation including imaging collection (including CT/MRI of chest, abdomen, pelvis; whole body bone scan (if clinically indicated); bone x-ray; CT or MRI (for bone lesions only if present at screening); brain CT/MRI (for brain metastasis if present at screening); skin color photography (if skin lesions present at screening); and any other imaging methods if existing or suspected lesions at screening). Except for whole body bone scan and other imaging methods, all other imaging collections are conducted every 8 weeks (+/−7 days).

In addition, safety is monitored by assessing physical examination, vital signs, height and weight, performance status, laboratory evaluations, cardiac assessments, adverse events, surgical/medical procedures and concomitant medications.

Pharmacokinetics of the study drugs are also analyzed.

Available clinical data is analyzed after each cohort of evaluable patients has completed the first 35 days of treatment for decision-making on the dose for the next cohort and/or for determining the maximal tolerated dose (MTD)/recommended dose for expansion (RDE). One interim analysis to support the declared MTD/RDE is performed after all patients enrolled in the dose escalation phase have completed day-35 or have discontinued study treatment.

After discontinuation of treatment, the end of treatment visit is occurring within 14 days after the last administration of study treatment. The end-of-treatment visit includes a full assessment of physical examination, laboratory assessments, imaging, and safety.

All patients are provided with safety evaluations for 30 days after the last dose of study treatment. Patients whose treatment is interrupted or permanently discontinued due to an adverse event, including abnormal laboratory value, must be followed at least once a week for 4 weeks and subsequently at 4 week intervals until resolution or stabilization of the event, whichever comes first.

All patients who do not discontinue study treatment due to disease progression, death, start of new anti-neoplastic therapies, lost to follow-up, or withdrawal of consent to efficacy follow-up are provided with efficacy follow-up. During efficacy follow-up, tumor evaluations (including CT/MRI of chest, abdomen, pelvis), whole body bone scan (if clinically indicated), bone x-ray, CT or MRI (for bone lesions only if present at screening), brain CT/MRI (for brain metastasis if present at screening), skin color photography (if skin lesions present at screening), and any other imaging methods if existing or suspected lesions at screening) are continued with an evaluation every 8 weeks (+/−7 days). In addition, antineoplastic therapies administered to the patient are documented.

All patients are followed for survival status every 3 months regardless of treatment discontinuation reason until death, lost to follow-up, or withdrawal of consent to survival follow-up. Additional survival assessments may be performed outside the 3-month follow-up schedules if a survival update is required for an interim assessment to meet safety or regulatory needs.

The study is ending when either all patients are deceased, or have completed the study treatment and at least 6 months survival follow-up, or have been lost to follow-up or withdrew consent, whichever occurs first. An initial clinical safety and efficacy assessment may be conducted after all patients have completed 6 cycles of treatment. The final clinical assessment is conducted at the end of the study.

It is understood that the benefits of the triple combination comprising COMPOUND A, everolimus and exemestane may be assessed either during the study treatment period or at the end of the study.

What is claimed is:

1. A pharmaceutical combination comprising: (a) an alpha-isoform specific phosphatidylinositol-3-kinase (PI3K) inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor everolimus or any pharmaceutically acceptable salt thereof, and (c) exemestane or any pharmaceutically acceptable salt thereof.

2. A pharmaceutical combination according to claim 1, for simultaneous, separate or sequential use in the treatment of a proliferative disease.

3. A pharmaceutical combination according to claim 2, wherein the proliferative disease is a cancer.

4. A pharmaceutical combination according to claim 2, wherein the proliferative disease is a hormone-receptor positive breast cancer.

5. A method for treating a proliferative disease in a subject in need thereof comprising administering to said subject a therapeutically effective amount of (a) an alpha-isoform specific phosphatidylinositol-3-kinase (PI3K) inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, (b) an mTOR inhibitor everolimus or any pharmaceutically acceptable salt thereof, and (c) exemestane or any pharmaceutically acceptable salt thereof.

6. A method according to claim 5, wherein the proliferative disease is a cancer.

7. A method according to claim 5, wherein the proliferative disease is breast cancer, pancreatic neuroendocrine tumor or renal cell carcinoma.

8. A method according to claim 5, wherein the proliferative disease is hormone-receptor positive breast cancer.

9. A commercial package comprising as active ingredient an alpha-isoform specific phosphatidylinositol-3-kinase (PI3K) inhibitor inhibitor (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and instructions for simultaneous, separate or sequential administration of said active ingredient with (a) an mTOR inhibitor everolimus or any pharmaceutically acceptable salt thereof and (b) exemestane or any pharmaceutically acceptable salt thereof to a patient in need thereof for use in the treatment of a proliferative disease.
